(19) United States
(12) Patent Application Publication
(10) Pub. No.: US 2017/0007215 A1
(43) Pub. Date: Jan. 12, 2017

(54) SYSTEM FOR SALIVA COLLECTION

(71) Applicant: SALVME, INC., East Palo Alto, CA (US)

(72) Inventor: Erez Podoly, Menlo Park, CA (US)

(73) Assignee: SALVME, INC., East Palo Alto, CA (US)

(21) Appl. No.: 15/115,386

(22) PCT Filed: Jan. 29, 2015
(86) PCT No.: PCT/US15/13594

§ 371 Date: Jul. 29, 2016

Related U.S. Application Data
(60) Provisional application No. 61/933,816, filed on Jan. 30, 2014.

(51) Int. Cl. A61B 10/00; A61C 17/22; A61C 17/06; G01N 33/487; A61B 5/16; A61B 5/00; A61B 5/02; A61L 2/10; A61B 5/145; A61C 19/04

(52) U.S. Cl. CPC A61B 10/0051; A61B 5/14507; A61B 5/14532; A61B 5/14546; A61C 17/222; A61C 17/046; A61C 17/227; A61C 19/04; A61B 5/165; A61B 5/4547; A61B 5/4552; A61B 5/02028; A61B 5/425; A61B 5/4381; A61B 5/4815; A61B 5/4866; A61B 10/0012; A61B 10/0041; A61L 2/10; G01N 33/487; A61L 2202/11

(57) ABSTRACT

Disclosed herein are portable handheld devices for saliva collection for daily monitoring of salivary biomarkers of physiological conditions, the device comprising: a handheld handle comprising a saliva collector, the saliva collector comprising: a suction tip; a bi-directional pump electrically configurable for pumping towards two opposite directions; a saliva reservoir; a valve; a water reservoir, the water reservoir comprising an external filling lid; a reversible fluidic connection configurable in a connected and disconnected position; and a base accommodating the handheld handle, the base configurable to fluidly communicate with the handheld handle through at least the reversible fluidic connection, wherein the valve is electrically configured to allow fluid communication directly between at least two selected from: the saliva reservoir, the water reservoir, and the reversible fluidic connection, and wherein the bi-directional pump is electrically configurable to allow fluid communication directly between the suction tip and the saliva reservoir.

### CROSS-REFERENCE

This application claims the benefit of U.S. Provisional Application No. 61/933,816, filed Jan. 30, 2014, which is hereby incorporated by reference in its entirety.

### BACKGROUND OF THE INVENTION

Daily monitoring, early detection, prediction, and prevention of health problems are of significant values to the wellbeing of individuals and healthcare professionals. Existing salivary biomarker testing systems typically involves a swab placed between the lower cheek and gum for about two minutes, once saturated with saliva is transferred to a collection vial. Most tests are done at a healthcare facility followed by processing at a remote laboratory. Like other laboratory tests, the proper collection of a saliva sample is vital to an accurate result. Saliva testing may also becoming part of routine dental or medical office examinations where saliva collection is simple to perform.

Field of the Invention

The present invention relates to saliva collecting and saliva monitoring portable oral devices including saliva-monitoring electrical toothbrushes.

### SUMMARY OF THE INVENTION

Existing salivary biomarker testing systems with or without transportation procedures are inconvenient for patients and not suitable for daily or frequent usage. Furthermore, current systems are time-consuming and not suitable for protein biomarkers with short half-life and temperature sensitivity. Consequently, current saliva collection and/or transportation procedures result in an inherent inaccuracy and inefficiency in biomarker analysis.

There is a rapidly-growing need for an easy-to-use and accurate portable platform that implements non-invasive automated monitoring of known salivary biomarkers of physical health chronic conditions. Home-based portable platforms of saliva collection and biomarker testing are much more convenient and time-efficient than their hospital-based equivalents since it can be used on a daily basis, and be easily conjugated with a daily routine, for example, tooth brushing. Testing results can be generated within the same home-based device to reflect instant fluctuation in health conditions and eliminates the unnecessary long processing wait times for laboratory results. Thus, possible health problems can be detected and addressed more quickly and effectively. In addition, the portable and easy-to-use features allow accumulation of a larger scale of data compared to existing solutions. As a result, testing using a home-based portable system can be more accurate, reliable, and capable of tracking time evolution of data—all necessary for early detection of diseases. When fine-tuned, the home-based portable platforms may be used to facilitate monitoring of drug compliance, sending patients to immediate consultation, rushing patients to the Emergency Room (ER), alerting patients' caregivers and family members and save patients' lives. The platform will potentially reduce death-cases numbers and severity of disease conditions, number of unnecessary ER and clinic visits and consequently healthcare costs.

In one aspect, discloses herein are bi-functional portable handheld devices for tooth brushing and saliva collection for daily monitoring of salivary biomarkers of physiological conditions of a subject, the device comprising: a toothbrush comprising: a toothbrush head comprising a plurality of bristles, wherein the toothbrush head is configured to retract the plurality of bristles; a UV light source configured to disinfect the retracted plurality of bristles; a handheld handle comprising a saliva collector, the saliva collector comprising: a suction tip; a bi-directional pump electrically configurable for pumping towards two opposite directions; a saliva reservoir; a valve; a water reservoir, the water reservoir comprising an external filling lid; a reversible fluidic connection configurable in a connected position and a disconnected position; and a base accommodating the handheld handle, the base configurable to fluidly communicate with the handheld handle through at least the reversible fluidic connection, wherein the valve is electrically configured to allow fluid communication directly between at least two selected from: the saliva reservoir, the water reservoir, and the reversible fluidic connection, and wherein the bi-directional pump is electrically configurable to allow fluid communication directly between the suction tip and the saliva reservoir.

In another aspect, discloses herein are uni-functional portable handheld devices for tooth brushing and saliva collection for daily monitoring of salivary biomarkers of physiological conditions of a subject, the device comprising: a handheld handle comprising a saliva collector, the saliva collector comprising: a suction tip; a bi-directional pump electrically configurable for pumping towards two opposite directions; a saliva reservoir; a valve; a water reservoir, the water reservoir comprising an external filling lid; a reversible fluidic connection configurable in a connected position and a disconnected position; and a base accommodating the handheld handle, the base configurable to fluidly communicate with the handheld handle through at least the reversible fluidic connection, wherein the valve is electrically configured to allow fluid communication directly between at least two selected from: the saliva reservoir, the water reservoir, and the reversible fluidic connection, and wherein the bi-directional pump is electrically configurable to allow fluid communication directly between the suction tip and the saliva reservoir.

In another aspect, discloses herein are methods of saliva collection using a portable handheld device for daily monitoring of at salivary biomarkers of physiological conditions of a subject, the method comprising: rinsing a handheld handle of the device using a bi-directional pump using water or a rinsing solution from a water reservoir, the handheld handle of the device being properly detached from a base accommodating the device, wherein a valve is in an open position and a reversible fluidic connection is in a connected position; collecting a saliva sample from a mouth of the subject through a suction tip to a saliva reservoir; and transporting the saliva sample from the saliva reservoir to the base, the handheld handle being properly placed back in the base and the fluidic connection between the handheld handle and the base being electrically established, wherein the valve is in the open position and the reversible fluidic connection is in the connected position; wherein the valve is electrically configured to allow fluid communication directly between at least two selected from: the saliva reservoir, the water reservoir, and the reversible fluidic connection, and wherein the bi-directional pump is electrically configurable to allow fluid communication directly between the suction tip and the saliva reservoir, and wherein the water reservoir comprises an external filling lid.

In another aspect, discloses herein are bi-functional portable handheld devices for tooth brushing and saliva collection for daily monitoring of physiological conditions of a subject, the device comprising: a toothbrush comprising: a toothbrush head comprising a plurality of bristles, wherein the toothbrush head is configured to retract the plurality of bristles; a UV light source configured to disinfect the retracted plurality of bristles; and a handheld handle comprising a saliva collector and ejector, the saliva collector and ejector comprising: a first valve electrically configurable in an open position and a closed position; a suction tip; a saliva reservoir, a bi-directional pump electrically configurable for a withdrawal, an ejection, and a stop; a reversible fluidic connection configurable in a connected position and a disconnected position, wherein the first valve is fluidly connected directly to the saliva reservoir, the suction tip, the bi-directional pump, and the reversible fluidic connection, and wherein the saliva reservoir is fluidly connected directly to the bi-directional pump.

In another aspect, disclosed herein are bi-functional portable handheld devices for saliva collection for daily monitoring of physiological conditions of a subject, the device comprising: a handheld handle comprising a saliva collector and ejector, the saliva collector and ejector comprising: a first valve electrically configurable in an open position and a closed position; a suction tip; a saliva reservoir, a bi-directional pump electrically configurable for a withdrawal, an ejection, and a stop; a reversible fluidic connection configurable in a connected position and a disconnected position, wherein the first valve is fluidly connected directly to the saliva reservoir, the suction tip, the bi-directional pump, and the reversible fluidic connection, and wherein the saliva reservoir is fluidly connected directly to the bi-directional pump.

In another aspect, discloses herein are bi-functional portable handheld devices for tooth brushing, saliva collection, and saliva testing for daily monitoring of physiological conditions of a subject, the device comprising a toothbrush comprising: a toothbrush head comprising a plurality of bristles, wherein the toothbrush head is configured to retract the plurality of bristles; a UV light source configured to disinfect the retracted plurality of bristles; a handheld handle comprising a saliva collector and ejector, the saliva collector and ejector comprising: a first valve electrically configurable in an open position and a closed position; a second valve electrically configurable in an open position and a closed position; a suction tip; a saliva reservoir; a water reservoir; a bi-directional pump electrically configurable for a withdrawal, an ejection, and a stop; a reversible fluidic connection configurable in a connected position and a disconnected position; and a base accommodating the handheld device, the base comprising: a detection unit configurable to connect or disconnect to the reversible fluidic connection; a buffer reservoir configurable to fluidly connect or disconnect to the detection unit; a disposable cartridge configurable to fluidly connect to the detection unit, wherein the first valve is fluidly connected directly to the saliva reservoir, the suction tip, the second valve, and wherein the second valve is fluidly connected directly to the water reservoir, the saliva reservoir, and the reversible fluidic connection, and wherein the saliva reservoir is fluidly connected directly to the bi-directional pump.

In another aspect, discloses herein are bi-functional portable handheld devices for saliva collection, and saliva testing for daily monitoring of physiological conditions of a subject, the device comprising a handheld handle comprising a saliva collector and ejector, the saliva collector and ejector comprising: a first valve electrically configurable in an open position and a closed position; a second valve electrically configurable in an open position and a closed position; a suction tip; a saliva reservoir; a water reservoir; a bi-directional pump electrically configurable for a withdrawal, an ejection, and a stop; a reversible fluidic connection configurable in a connected position and a disconnected position; and a base accommodating the handheld device, the base comprising: a detection unit configurable to connect or disconnect to the reversible fluidic connection; a buffer reservoir configurable to fluidly connect or disconnect to the detection unit; a disposable cartridge configurable to fluidly connect to the detection unit, wherein the first valve is fluidly connected directly to the saliva reservoir, the suction tip, the second valve, and wherein the second valve is fluidly connected directly to the water reservoir, the saliva reservoir, and the reversible fluidic connection, and wherein the saliva reservoir is fluidly connected directly to the bi-directional pump.

In another aspect, disclosed herein are methods of saliva collection and testing using a portable handheld device for daily monitoring of physiological conditions of a subject, the method comprising: rinsing the device using a bi-directional pump using liquid from at least one selected from a saliva reservoir, a water reservoir, a suction tip, and a base accommodating the device, wherein a first valve and a second valve are in an open position and a reversible fluidic connection is in a connected position; collecting saliva from the suction tip to the saliva reservoir, wherein the first valve is in the open position; and transmitting the saliva from the saliva reservoir to a detection unit enclosed in the base, wherein the second valve is in the open position and the reversible fluidic connection is in the connected position; testing the saliva for at least one biomarker in the detection unit; wherein the first valve is fluidly connected directly to the saliva reservoir, the suction tip, the second valve, and wherein the second valve is fluidly connected directly to the water reservoir, the saliva reservoir, and the reversible fluidic connection, and wherein the saliva reservoir is fluidly connected directly to the bi-directional pump, and the reversible fluidic connection is fluidly connected to the detection unit.

### BRIEF DESCRIPTION OF THE DRAWINGS

FIG. 1 shows a non-limiting example of an electrical handheld portable device for saliva-collection and tooth brushing and a base accommodating the device.

FIG. 2 shows a non-limiting example of an electrical handheld portable device for saliva-collection and a base accommodating the device.

FIG. 3 shows a non-limiting example of a toothbrush head of a portable device for saliva collection and tooth brushing.

FIG. 4 shows a non-limiting example of a reversible fluidic connection of the electrical handheld portable device.

FIG. 5 shows a non-limiting example of internal connections of a handheld handle of a portable device for saliva collection.

FIG. 6a shows a non-limiting example of internal connections of a handheld handle of a portable device for saliva collection.

FIG. 6b shows a non-limiting example of internal connections of a handheld handle of a portable device for saliva collection.

FIG. 7 shows a non-limiting example of internal connections of a handheld handle of a portable device for saliva collection.

### DETAILED DESCRIPTION OF THE INVENTION

Existing salivary biomarker testing systems with or without transportation procedures are inconvenient for patients and not suitable for daily or frequent usage. Furthermore, current systems are time-consuming and not suitable for protein biomarkers with short half-life and temperature sensitivity. Consequently, current saliva collection and/or transportation procedures result in an inherent inaccuracy and inefficiency in biomarker analysis.

There is a rapidly-growing need for an easy-to-use and accurate portable platform that implements non-invasive automated monitoring of known salivary biomarkers of physical health chronic conditions. Home-based portable platforms of saliva collection and biomarker testing are much more convenient and time-efficient than their hospital-based equivalents since it can be used on a daily basis, and be easily conjugated with a daily routine, for example, tooth brushing. Testing results can be generated within the same home-based device to reflect instant fluctuation in health conditions and eliminates the unnecessary long processing wait times for laboratory results. Thus, possible health problems can be detected and addressed more quickly and effectively. In addition, the portable and easy-to-use features allow accumulation of a larger scale of data compared to existing solutions. As a result, testing using a home-based portable system can be more accurate, reliable, and capable of tracking time evolution of data—all necessary for early detection of diseases. When fine-tuned, the home-based portable platforms may be used to facilitate monitoring of drug compliance, sending patients to immediate consultation, rushing patients to the Emergency Room (ER), alerting patients' caregivers and family members and save patients' lives. The platform will potentially reduce death-cases numbers and severity of disease conditions, number of unnecessary ER and clinic visits and consequently healthcare costs.

Described herein, in certain embodiments, are bi-functional portable handheld devices for tooth brushing and saliva collection for daily monitoring of salivary biomarkers of physiological conditions of a subject, the device comprising: a toothbrush comprising: a toothbrush head comprising a plurality of bristles, wherein the toothbrush head is configured to retract the plurality of bristles; a UV light source configured to disinfect the retracted plurality of bristles; a handheld handle comprising a saliva collector, the saliva collector comprising: a suction tip; a bi-directional pump electrically configurable for pumping towards two opposite directions; a saliva reservoir; a valve; a water reservoir, the water reservoir comprising an external filling lid; a reversible fluidic connection configurable in a connected position and a disconnected position; and a base accommodating the handheld handle, the base configurable to fluidly communicate with the handheld handle through at least the reversible fluidic connection, wherein the valve is electrically configured to allow fluid communication directly between at least two selected from: the saliva reservoir, the water reservoir, and the reversible fluidic connection, and wherein the bi-directional pump is electrically configurable to allow fluid communication directly between the suction tip and the saliva reservoir. In some embodiments, the toothbrush is configured to retract the plurality of bristles mechanically or electrically. In some embodiments, the toothbrush comprises a replaceable platform configured to allow replacement of the toothbrush head. In some embodiments, the toothbrush head is configured to put the plurality of bristles into motion. In some embodiments, the motion of the plurality of bristles comprises one or more selected from: oscillation, vibration, translation, and rectilinear movement. In some embodiments, the UV light source is at least one UV LED. In some embodiments, the UV light source is located on the inner surface of the toothbrush head. In some embodiments, the direct fluidic communication is through at least one internal tube. In some embodiments, the direct fluidic communication is bi-directional. In some embodiments, the suction tip comprises a filter. In some embodiments, the saliva collector is configured to not use a capillary force. In some embodiments, the saliva reservoir is configured to temporally store a saliva sample therewithin. In some embodiments, a sample within the saliva reservoir is automatically transported to the base when the handheld handle is properly placed back in the base and the fluidic connection between the handheld handle and the base is electrically established. In some embodiments, the saliva reservoir is configurable to be hermetically sealed from at least one selected from: the bi-directional pump, the suction tip, the reversible fluidic connection, the base, the valve, and the water reservoir. In some embodiments, the valve is a three-way electrical valve. In some embodiments, the valve is a four-way electrical valve. In some embodiments, the external filling lid is not obstructed by the base. In some embodiments, n the external filling lid is configurable to be hermetically sealable and openable. In some embodiments, the water reservoir is located higher than the saliva reservoir in a vertical direction. In some embodiments, the water reservoir is configurable to enclose water or saliva rinsing solution therewithin. In some embodiments, the water reservoir is sized to allow at least two successful saliva rinsing steps without any refill of the water reservoir. In some embodiments, the reversible fluidic connection comprises a first magnet on the device and a second magnet on a base accommodating the device. In some embodiments, the devices, systems, and methods comprise a rechargeable power source. In some embodiments, the devices, systems, and methods comprise an electrical control unit. In some embodiments, the devices, systems, and methods comprise a user interface configured to allow user selection of at least one function selected from: saliva collection, saliva rinsing, saliva testing, tooth brushing, bristle retraction, bristle advancement, or bristle disinfection. In some embodiments, the devices, systems, and methods comprise a user interface configured to allow user selection of at least one preprogrammed sequence of functions selected from: saliva rinsing, and then saliva collection; saliva rinsing, then saliva collection, and then tooth brushing; saliva rinsing, then saliva collection, then tooth brushing, and then saliva testing. In some embodiments, the handheld handle comprises at least one connection interface configurable to connect to the suction tip but not connect to the toothbrush head. In some embodiments, the handheld handle comprises at least one connection interface configurable to connect to the toothbrush head. In some embodiments, the base is fluidly connected to the handheld handle automatically via the reversible fluidic connection when the handheld handle is properly placed back in the base and at least one predetermined condition is met. In some embodiments, the base is electrically connected to the device automatically when the handheld handle is properly placed back in the base. In some embodiments, the base comprises an outlet or a dispenser, wherein the outlet or the dispenser is fluidly connected to the reversible fluidic connection. In some embodiments, the base comprises a power source. In some embodiments, the base comprises: a second reversible fluidic connection configurable in a connected position and a disconnected position; a detection unit configurable to fluidly communicate with the second reversible fluidic connection; a buffer reservoir configurable to fluidly communicate with the detection unit; a second electrical pump; a port configurable to hold a removable cartridge and allow fluidic connection of the removable cartridge to the detection unit; a microprocessor; and a wireless system, wherein the second reversible fluidic connection is configured to allow fluidic connection to the handheld handle. In some embodiments, the base is fluidly connected to the handheld handle when the second reversible fluidic connection and the reversible fluidic connection of the handheld handle are in the connected position. In some embodiments, the base is fluidly connected to the handheld handle when the handheld handle is properly placed back in the base and a predetermined condition is met. In some embodiments, the removable cartridge is configurable for dispensing liquid. In some embodiments, the removable cartridge is configurable to hold at least one reagent therewithin. In some embodiments, the wireless system is configurable to transmit data to or receive data from at least one external source. In some embodiments, the device is configurable to monitor at least one salivary biomarker for at least one physiological condition in at least one category selected from: sleep quality, physical performance, fertility-related conditions, oral-related diseases, bone, nutrition, autoimmune diseases, cardiovascular health systemic diseases, psychological health, and common cancer types. In some embodiments, the device is configurable to monitor at least one salivary biomarker for at least one physiological condition selected from: sleep apnea, sleep deprivation, jet lag recovery, fatigue, overtraining syndrome, ovulation, pregnancy, menopause, gingivitis, oral carcinoma, squamous cell carcinoma, OSCC, osteoporosis, alveolar remodeling, glucose abnormalities, metabolic/anabolic status, eating disorders, rheumatoid arthritis, Sjogren's syndrome, lymphoepithelial lesion, acute myocardial infarction, diabetes, fibromyalgia, gout, mood disorder, depression, suicidality, anxiety, stress, breast cancer, prostate cancer, pancreatic cancer, lung-and-bronchus cancer, and colorectal cancer. In some embodiments, the device is configurable to monitor at least one salivary biomarker selected from: glucose, fatty acid, nitric oxide, cortisol, c-reactive protein (CRP), secretory immunoglobulin A (IgA), solute carrier family 6 (SLC6A4), 5-hydroxytryptamine-2C (5HT2C), gated calcium channel, ankyrin G, a1-anti-trypsin, brain-derived neurotrophic factor (BDNF), calcium binding protein B (S100B), myelo-peroxidase, soluble tumor necrosis factor receptor type II (s-TNFRII), epidermal growth factor (EGF), prolactin, resistin, senescence-associated secretory phenotype (SASP), spermidine/spermine N1-acetyltransferase (1SAT1), phosphatase and tensin homolog (PTEN), myristoylated alanine-rich C-kinase substrate (MARCKS), mitogen-activated protein kinase-kinase-kinase-3 (MAP3K3), signal transducer cluster of differentiation 24/heat stable antigen (CD24/HSA), forkhead box protein N3 (FOXN3), phosphoinositide-3-kinase, regulatory subunit 5 (PIK3R5), guanine nucleotide-binding protein 1 (GBP1), calcitonin gene related peptide (CGRP), estrogen, vasoactive intestinal peptide VIP, alpha-amylase, prostaglandin E2 (PGE2), prostacyclin (PGI2), β-endorphin, corticotropin-releasing hormone (CRH), dehydroepiandrosterone (DHEA), dehydroepiandrosterone-sulfate (DHEA-S), estradiol, interleukin-lbeta (IL-1β), interleukin 2 (IL-2), interleukin 4 (IL-4), interleukin 5 (IL-5), interleukin 6 (IL-6), interleukin 9 (IL-9), interleukin 17 (IL-17), progesterone, tumor necrosis factor alpha (TNF-α); cardiac troponin I (cTnI) cardiac troponin T (cTnT), creatine phosphokinase-MB (CK-MB), brain natriuretic peptide (BNP), N-terminal pro-brain natriuretic peptide (NT-proBNP); myoglobin, matrix metallopeptidase 9 (MMP-9), TNF receptor superfamily member 5 (CD40), soluble intercellular adhesion molecule-1 (sICAM-1), breast cancer 1 and 2 (BRCA1&2), receptor tyrosine-protein kinase erbB-2/human epidermal growth factor receptor2 (C-erbB-2/HER2/neu), cancer antigen 15-3 (CA15-3), mucin 1, cathepsin D, p53, homeodomain-containing transcription factor NKX3.1, zinc-alpha-2-glycoprotein, calprotectin (S100A8, S100A9), anaplastic lymphoma kinase (ALK), epidermal growth factor receptor (EGFR), V-Ki-ras2 Kirsten rat sarcoma viral oncogene homolog (KRAS), v-Raf murine sarcoma viral oncogene homolog B (braf V600E), carcinoembryonic antigen (CEA), uridine diphosphate glucuronyltransferase 1-1 (UGT1-A1), 3-methylhistidine, glucose-1-phosphate, glucose-6-phosphate, taurine, chromogranin A, melatonin, testosterone, 1,5-anhydroglucitol, chromogranin A, oxido-reductase; glycated hemoglobin (HbAlc), uric acid, transaldolase, phosphoglycerate mutase I, luteinizing hormone (LH), estrone-3-glucuronide (E3G), follicle stimulating hormone (FSH), follicle stimulating hormone (FSH), Androstenedione, Estradiol (E2), Estriol (E3), 17α-hydroxyprogesterone, androstenedione, estrogen receptors, estrone glucuronide (E1G) pregnanediol glucuronide (PdG), androstenedione, insulin-like growth factor 1 (IGF-1), tau, amyloid beta, alpha-synuclein, parkinson protein 7 (PARK7), human leucocyte antigens (HLAs) type II, fractalkine.

Described herein, in certain embodiments, are uni-functional portable handheld devices for tooth brushing and saliva collection for daily monitoring of salivary biomarkers of physiological conditions of a subject, the device comprising: a handheld handle comprising a saliva collector, the saliva collector comprising: a suction tip; a bi-directional pump electrically configurable for pumping towards two opposite directions; a saliva reservoir; a valve; a water reservoir, the water reservoir comprising an external filling lid; a reversible fluidic connection configurable in a connected position and a disconnected position; and a base accommodating the handheld handle, the base configurable to fluidly communicate with the handheld handle through at least the reversible fluidic connection, wherein the valve is electrically configured to allow fluid communication directly between at least two selected from: the saliva reservoir, the water reservoir, and the reversible fluidic connection, and wherein the bi-directional pump is electrically configurable to allow fluid communication directly between the suction tip and the saliva reservoir. In some embodiments, the devices, systems, and methods comprise a toothbrush comprising: a toothbrush head comprising a plurality of bristles, wherein the toothbrush head is configured to retract the plurality of bristles; and a UV light source configured to disinfect the retracted plurality of bristles. In some cases, the toothbrush is configured to retract the plurality of bristles mechanically or electrically. In some cases, the toothbrush comprises a replaceable platform configured to allow replacement of the toothbrush head. In some cases, the toothbrush head is configured to put the plurality of bristles into motion. In some cases, the motion of the plurality of bristles comprises one or more selected from: oscillation, vibration, translation, and rectilinear movement. In some cases, the UV light source is at least one UV LED. In some cases, the UV light source is located on the inner surface of the toothbrush head. In some cases, the direct fluidic communication is through at least one internal tube. In some cases, the direct fluidic communication is bi-directional. In some cases, the suction tip comprises a filter. In some cases, the saliva collector is configured to not use a capillary force. In some cases, the saliva reservoir is configured to temporally store a saliva sample therewithin. In some cases, a sample within the saliva reservoir is automatically transported to the base when the handheld handle is properly placed back in the base and the fluidic connection between the handheld handle and the base is electrically established. In some cases, the saliva reservoir is configurable to be hermetically sealed from at least one selected from: the bi-directional pump, the suction tip, the reversible fluidic connection, the base, the valve, and the water reservoir. In some cases, the valve is a three-way electrical valve. In some cases, the valve is a four-way electrical valve. In some cases, the external filling lid is not obstructed by the base. In some cases, the external filling lid is configured to be hermetically sealable. In some cases, the water reservoir is located higher than the saliva reservoir in a vertical direction. In some cases, the water reservoir is configurable to enclose water or saliva rinsing solution therewithin. In some cases, the water reservoir is sized to allow at least two successful saliva rinsing steps without any refill of the water reservoir. In some cases, the reversible fluidic connection comprises a first magnet on the device and a second magnet on a base accommodating the device. In some embodiments, the devices, systems, and methods comprise a rechargeable power source. In some embodiments, the devices, systems, and methods comprise a user interface configured to allow user selection of at least one function selected from: saliva collection, saliva rinsing, saliva transportation, saliva testing, tooth brushing, bristle retraction, bristle advancement, or bristle disinfection. In some embodiments, the devices, systems, and methods comprise a user interface configured to allow user selection of at least one preprogrammed sequence of functions selected from: saliva rinsing, then saliva collection, and then saliva transportation; saliva rinsing, then saliva collection, then saliva transportation, and then tooth brushing; or saliva rinsing, then saliva collection, then tooth brushing, and then saliva transportation. In certain cases, the handheld handle comprises at least one connection interface configurable to connect to the suction tip but not connect to the toothbrush head. In certain cases, the handheld handle comprises at least one connection interface configurable to connect to the toothbrush head. In certain cases, the base is fluidly connected to the handheld handle automatically via the reversible fluidic connection when the handheld handle is properly placed back in the base and a predetermined condition is met. In certain cases, the base is electrically connected to the device automatically when the handheld handle is properly placed back in the base. In certain cases, the base comprises an outlet or a dispenser, wherein the outlet or the dispenser is fluidly connected to the reversible fluidic connection. In certain cases, the base comprises a power source. In some embodiments, the base is fluidly connected to the handheld handle automatically via the reversible fluidic connection when the handheld handle is properly placed back in the base and at least one predetermined condition is met. In some embodiments, the base is electrically connected to the device automatically when the handheld handle is properly placed back in the base. In some embodiments, the base comprises an outlet or a dispenser, wherein the outlet or the dispenser is fluidly connected to the reversible fluidic connection. In some embodiments, the base comprises a power source. In some embodiments, the base comprises: a second reversible fluidic connection configurable in a connected position and a disconnected position; a detection unit configurable to fluidly communicate with the second reversible fluidic connection; a buffer reservoir configurable to fluidly communicate with the detection unit; a second electrical pump; a port configurable to hold a removable cartridge and allow fluidic connection of the removable cartridge to the detection unit; a microprocessor; and a wireless system, wherein the second reversible fluidic connection is configured to allow fluidic connection to the handheld handle. In some embodiments, the base is fluidly connected to the handheld handle when the second reversible fluidic connection and the reversible fluidic connection of the handheld handle are in the connected position. In some embodiments, the base is fluidly connected to the handheld handle when the handheld handle is properly placed back in the base and a predetermined condition is met. In some embodiments, the removable cartridge is configurable for dispensing liquid. In certain cases, the device is configurable to monitor at least one salivary biomarker for at least one physiological condition in at least one category selected from: sleep quality, physical performance, fertility-related conditions, oral-related diseases, bone, nutrition, autoimmune diseases, cardiovascular health systemic diseases, psychological health, and common cancer types. In certain cases, the device is configurable to monitor at least one salivary biomarker for at least one physiological condition selected from: sleep apnea, sleep deprivation, jet lag recovery, fatigue, overtraining syndrome, ovulation, pregnancy, menopause, gingivitis, oral carcinoma, squamous cell carcinoma, OSCC, osteoporosis, alveolar remodeling, glucose abnormalities, metabolic/anabolic status, eating disorders, rheumatoid arthritis, Sjogren's syndrome, lymphoepithelial lesion, acute myocardial infarction, diabetes, fibromyalgia, gout, mood disorder, depression, suicidality, anxiety, stress, breast cancer, prostate cancer, pancreatic cancer, lung-and-bronchus cancer, and colorectal cancer. In certain cases, the device is configurable to monitor at least one salivary biomarker selected from: glucose, fatty acid, nitric oxide, cortisol, c-reactive protein (CRP), secretory immunoglobulin A (IgA), solute carrier family 6 (SLC6A4), 5-hydroxytryptamine-2C (5HT2C), gated calcium channel, ankyrin G, a1-anti-trypsin, brain-derived neurotrophic factor (BDNF), calcium binding protein B (S100B), myelo-peroxidase, soluble tumor necrosis factor receptor type II (s-TNFRII), epidermal growth factor (EGF), prolactin, resistin, senescence-associated secretory phenotype (SASP), spermidine/spermine N1-acetyltransferase (1SAT1), phosphatase and tensin homolog (PTEN), myristoylated alanine-rich C-kinase substrate (MARCKS), mitogen-activated protein kinase-kinase-kinase-3 (MAP3K3), signal transducer cluster of differentiation 24/heat stable antigen (CD24/HSA), forkhead box protein N3 (FOXN3), phosphoinositide-3-kinase, regulatory subunit 5 (PIK3R5), guanine nucleotide-binding protein 1 (GBP1), calcitonin gene related peptide (CGRP), estrogen, vasoactive intestinal peptide VIP, alpha-amylase, prostaglandin E2 (PGE2), prostacyclin (PGI2), β-endorphin, corticotropin-releasing hormone (CRH), dehydroepiandrosterone (DHEA), dehydroepiandrosterone-sulfate (DHEA-S), estradiol, interleukin-lbeta (IL-1β), interleukin 2 (IL-2), interleukin 4 (IL-4), interleukin 5 (IL-5), interleukin 6 (IL-6), interleukin 9 (IL-9), interleukin 17 (IL-17), progesterone, tumor necrosis factor alpha (TNF-α); cardiac troponin I (cTnI) cardiac troponin T (cTnT), creatine phosphokinase-MB (CK-MB), brain natriuretic peptide (BNP), N-terminal pro-brain natriuretic peptide (NT-proBNP); myoglobin, matrix metallopeptidase 9 (MMP-9), TNF receptor superfamily member 5 (CD40), soluble intercellular adhesion molecule-1 (sICAM-1), breast cancer 1 and 2 (BRCA1&2), receptor tyrosine-protein kinase erbB-2/human epidermal growth factor receptor2 (C-erbB-2/HER2/neu), cancer antigen 15-3 (CA15-3), mucin 1, cathepsin D, p53, homeodomain-containing transcription factor NKX3.1, zinc-alpha-2-glycoprotein, calprotectin (S100A8, S100A9), anaplastic lymphoma kinase (ALK), epidermal growth factor receptor (EGFR), V-Ki-ras2 Kirsten rat sarcoma viral oncogene homolog (KRAS), v-Raf murine sarcoma viral oncogene homolog B (braf V600E), carcinoembryonic antigen (CEA), uridine diphosphate glucuronyltransferase 1-1 (UGT1-A1), 3-methylhistidine, glucose-1-phosphate, glucose-6-phosphate, taurine, chromogranin A, melatonin, testosterone, 1,5-anhydroglucitol, chromogranin A, oxido-reductase; glycated hemoglobin (HbAlc), uric acid, transaldolase, phosphoglycerate mutase I, luteinizing hormone (LH), estrone-3-glucuronide (E3G), follicle stimulating hormone (FSH), follicle stimulating hormone (FSH), Androstenedione, Estradiol (E2), Estriol (E3), 17α-hydroxyprogesterone, androstenedione, estrogen receptors, estrone glucuronide (E1G) pregnanediol glucuronide (PdG), androstenedione, insulin-like growth factor 1 (IGF-1), tau, amyloid beta, alpha-synuclein, parkinson protein 7 (PARK7), human leucocyte antigens (HLAs) type II, fractalkine.

Described herein, in certain embodiments, are methods of saliva collection using a portable handheld device for daily monitoring of at salivary biomarkers of physiological conditions of a subject, the method comprising: rinsing a handheld handle of the device using a bi-directional pump using water or a rinsing solution from a water reservoir, the handheld handle of the device being properly detached from a base accommodating the device, wherein a valve is in an open position and a reversible fluidic connection is in a connected position; collecting a saliva sample from a mouth of the subject through a suction tip to a saliva reservoir; and transporting the saliva sample from the saliva reservoir to the base, the handheld handle being properly placed back in the base and the fluidic connection between the handheld handle and the base being electrically established, wherein the valve is in the open position and the reversible fluidic connection is in the connected position; wherein the valve is electrically configured to allow fluid communication directly between at least two selected from: the saliva reservoir, the water reservoir, and the reversible fluidic connection, and wherein the bi-directional pump is electrically configurable to allow fluid communication directly between the suction tip and the saliva reservoir, and wherein the water reservoir comprises an external filling lid. In certain cases, the direct fluidic communication is through at least one internal tube. In certain cases, the direct fluidic communication is bi-directional. In certain cases, the suction tip comprises a filter. In certain cases, the saliva collector is configured to not use a capillary force. In certain cases, the saliva reservoir is configured to temporally store a saliva sample therewithin. In certain cases, a sample within the saliva reservoir is automatically transported to the base when the handheld handle is properly placed back in the base and the fluidic connection between the handheld handle and the base is electrically established. In certain cases, the saliva reservoir is configurable to be hermetically sealed from at least one selected from: the bi-directional pump, the suction tip, the reversible fluidic connection, the base, the valve, and the water reservoir. In certain cases, the valve is a three-way electrical valve. In certain cases, the valve is a four-way electrical valve. In certain cases, the external filling lid is not obstructed by the base. In certain cases, the external filling lid is configured to be hermetically sealable. In certain cases, the water reservoir is located higher than the saliva reservoir in a vertical direction. In certain cases, the water reservoir is configurable to enclose water or saliva rinsing solution therewithin. In certain cases, wherein the water reservoir is sized to allow at least two successful saliva rinsing steps without any refill of the water reservoir. In certain cases, the reversible fluidic connection comprises a first magnet on the device and a second magnet on a base accommodating the device. In some cases, the methods, systems, and devices disclosed herein comprise a rechargeable power source. In some cases, the methods, systems, and devices disclosed herein a user interface configured to allow user selection of at least one function selected from: saliva collection, saliva rinsing, saliva testing, saliva transportation, tooth brushing, bristle retraction, bristle advancement, or bristle disinfection. In some cases, the methods, systems, and devices disclosed herein a user interface configured to allow user selection of at least one preprogrammed sequence of functions selected from: saliva rinsing, then saliva collection, and then saliva transportation; saliva rinsing, then saliva collection, then saliva transportation, and then tooth brushing; or saliva rinsing, then saliva collection, then tooth brushing, and then saliva transportation. In some cases, the handheld handle comprises at least one connection interface configurable to connect to the suction tip but not connect to the toothbrush head. In some cases, the handheld handle comprises at least one connection interface configurable to connect to the toothbrush head. In some cases, the base is fluidly connected to the handheld handle automatically via the reversible fluidic connection when the handheld handle is properly placed back in the base and a predetermined condition is met. In some cases, the base is electrically connected to the device automatically when the handheld handle is properly placed back in the base. In some cases, the base comprises an outlet or a dispenser, wherein the outlet or the dispenser is fluidly connected to the reversible fluidic connection. In some cases, the base comprises a power source. In some embodiments, the base is fluidly connected to the handheld handle automatically via the reversible fluidic connection when the handheld handle is properly placed back in the base and at least one predetermined condition is met. In some embodiments, the base is electrically connected to the device automatically when the handheld handle is properly placed back in the base. In some embodiments, the base comprises an outlet or a dispenser, wherein the outlet or the dispenser is fluidly connected to the reversible fluidic connection. In some embodiments, the base comprises a power source. In some embodiments, the base comprises: a second reversible fluidic connection configurable in a connected position and a disconnected position; a detection unit configurable to fluidly communicate with the second reversible fluidic connection; a buffer reservoir configurable to fluidly communicate with the detection unit; a second electrical pump; a port configurable to hold a removable cartridge and allow fluidic connection of the removable cartridge to the detection unit; a microprocessor; and a wireless system, wherein the second reversible fluidic connection is configured to allow fluidic connection to the handheld handle. In some embodiments, the base is fluidly connected to the handheld handle when the second reversible fluidic connection and the reversible fluidic connection of the handheld handle are in the connected position. In some embodiments, the base is fluidly connected to the handheld handle when the handheld handle is properly placed back in the base and a predetermined condition is met. In some embodiments, the removable cartridge is configurable for dispensing liquid. In some cases, the method comprises filling the water reservoir with water or a rinsing solution. In some cases, rinsing the device stops when a pre-determined threshold has been met. In some cases, collecting the saliva sample stops when a pre-determined threshold has been met. In some cases, transporting the saliva sample is automatically activated when the handheld handle is properly placed back in the base and the fluidic connection between the handheld handle and the base is electrically established. In some cases, the device is configurable to monitor at least one salivary biomarker for at least one physiological condition in at least one category selected from: sleep quality, physical performance, fertility-related conditions, oral-related diseases, bone, nutrition, autoimmune diseases, cardiovascular health systemic diseases, psychological health, and common cancer types. In some cases, the device is configurable to monitor at least one salivary biomarker for at least one physiological condition selected from: sleep apnea, sleep deprivation, jet lag recovery, fatigue, overtraining syndrome, ovulation, pregnancy, menopause, gingivitis, oral carcinoma, squamous cell carcinoma, OSCC, osteoporosis, alveolar remodeling, glucose abnormalities, metabolic/anabolic status, eating disorders, rheumatoid arthritis, Sjogren's syndrome, lymphoepithelial lesion, acute myocardial infarction, diabetes, fibromyalgia, gout, mood disorder, depression, suicidality, anxiety, stress, breast cancer, prostate cancer, pancreatic cancer, lung-and-bronchus cancer, and colorectal cancer. In some cases, the device is configurable to monitor at least one salivary biomarker selected from: glucose, fatty acid, nitric oxide, cortisol, c-reactive protein (CRP), secretory immunoglobulin A (IgA), solute carrier family 6 (SLC6A4), 5-hydroxytryptamine-2C (5HT2C), gated calcium channel, ankyrin G, a1-anti-trypsin, brain-derived neurotrophic factor (BDNF), calcium binding protein B (S100B), myelo-peroxidase, soluble tumor necrosis factor receptor type II (s-TNFRII), epidermal growth factor (EGF), prolactin, resistin, senescence-associated secretory phenotype (SASP), spermidine/spermine N1-acetyltransferase (1SAT1), phosphatase and tensin homolog (PTEN), myristoylated alanine-rich C-kinase substrate (MARCKS), mitogen-activated protein kinase-kinase-kinase-3 (MAP3K3), signal transducer cluster of differentiation 24/heat stable antigen (CD24/HSA), forkhead box protein N3 (FOXN3), phosphoinositide-3-kinase, regulatory subunit 5 (PIK3R5), guanine nucleotide-binding protein 1 (GBP1), calcitonin gene related peptide (CGRP), estrogen, vasoactive intestinal peptide VIP, alpha-amylase, prostaglandin E2 (PGE2), prostacyclin (PGI2), β-endorphin, corticotropin-releasing hormone (CRH), dehydroepiandrosterone (DHEA), dehydroepiandrosterone-sulfate (DHEA-S), estradiol, interleukin-lbeta (IL-1β), interleukin 2 (IL-2), interleukin 4 (IL-4), interleukin 5 (IL-5), interleukin 6 (IL-6), interleukin 9 (IL-9), interleukin 17 (IL-17), progesterone, tumor necrosis factor alpha (TNF-α); cardiac troponin I (cTnI) cardiac troponin T (cTnT), creatine phosphokinase-MB (CK-MB), brain natriuretic peptide (BNP), N-terminal pro-brain natriuretic peptide (NT-proBNP); myoglobin, matrix metallopeptidase 9 (MMP-9), TNF receptor superfamily member 5 (CD40), soluble intercellular adhesion molecule-1 (sICAM-1), breast cancer 1 and 2 (BRCA1&2), receptor tyrosine-protein kinase erbB-2/human epidermal growth factor receptor2 (C-erbB-2/HER2/neu), cancer antigen 15-3 (CA15-3), mucin 1, cathepsin D, p53, homeodomain-containing transcription factor NKX3.1, zinc-alpha-2-glycoprotein, calprotectin (S100A8, S100A9), anaplastic lymphoma kinase (ALK), epidermal growth factor receptor (EGFR), V-Ki-ras2 Kirsten rat sarcoma viral oncogene homolog (KRAS), v-Raf murine sarcoma viral oncogene homolog B (braf V600E), carcinoembryonic antigen (CEA), uridine diphosphate glucuronyltransferase 1-1 (UGT1-A1), 3-methylhistidine, glucose-1-phosphate, glucose-6-phosphate, taurine, chromogranin A, melatonin, testosterone, 1,5-anhydroglucitol, chromogranin A, oxido-reductase; glycated hemoglobin (HbAlc), uric acid, transaldolase, phosphoglycerate mutase I, luteinizing hormone (LH), estrone-3-glucuronide (E3G), follicle stimulating hormone (FSH), follicle stimulating hormone (FSH), Androstenedione, Estradiol (E2), Estriol (E3), 17α-hydroxyprogesterone, androstenedione, estrogen receptors, estrone glucuronide (E1G) pregnanediol glucuronide (PdG), androstenedione, insulin-like growth factor 1 (IGF-1), tau, amyloid beta, alpha-synuclein, parkinson protein 7 (PARK7), human leucocyte antigens (HLAs) type II, fractalkine.

Described herein, in certain embodiments, are bi-functional portable handheld devices for tooth brushing, saliva collection, and saliva testing for daily monitoring of physiological conditions of a subject, the device comprising: a toothbrush comprising: a toothbrush head comprising a plurality of bristles, wherein the toothbrush head is configured to retract the plurality of bristles; a UV light source configured to disinfect the retracted plurality of bristles; and a handheld handle comprising a saliva collector and ejector, the saliva collector and ejector comprising: a first valve electrically configurable in an open position and a closed position; a suction tip; a saliva reservoir, a bi-directional pump electrically configurable for a withdrawal, an ejection, and a stop; a reversible fluidic connection configurable in a connected position and a disconnected position, wherein the first valve is fluidly connected directly to the saliva reservoir, the suction tip, the bi-directional pump, and the reversible fluidic connection, and wherein the saliva reservoir is fluidly connected directly to the bi-directional pump. In some embodiments, the toothbrush is configured to retract the plurality of bristles mechanically or electrically. In some embodiments, the toothbrush comprises a replaceable platform configured to allow replacement of the toothbrush head. In some embodiments, the toothbrush head is configured to put the plurality of bristles into motion. In further embodiments, the motion of the plurality of bristles comprises one or more selected from: oscillation, vibration, translation, and rectilinear movement. In some embodiments, the devices, methods, and systems disclosed herein comprise a second valve electrically configurable in the open position and the closed position. In further embodiments, the first valve, the second valve, and the reversible fluidic connection are configured to function independently. In further embodiments, an inlet configured to allow rinsing of the saliva collector and ejector, wherein the inlet is electrically controlled by the second valve. In further embodiments, the second valve is fluidly connected directly to the inlet, the saliva reservoir, the bi-directional pump, and the first valve. In further embodiments, the fluidic connection between the inlet and the second valve, the saliva reservoir and the second valve, the first valve and the suction tip, the first valve and the saliva reservoir, the saliva reservoir and the bi-directional pump, the first valve and the second valve, or the first valve and the reversible fluidic connection is via at least one internal tube. In further embodiments, the fluid connection between the inlet and the second valve, the saliva reservoir and the second valve, the first valve and the suction tip, the first valve and the saliva reservoir, the saliva reservoir and the bi-directional pump, the first valve and the second valve, or the first valve and the reversible fluidic connection is bi-directional. In some embodiments, the UV light source is at least one UV LED. In some embodiments, the suction tip comprises a filter. In some embodiments, the suction tip is a telescopic suction tip. In some embodiments, the reversible fluidic connection comprises a first magnet on the device and a second magnet on a base accommodating the device. In some embodiments, the first magnet and the second magnet provides an interfacial force in the range of 1 to 50 Newton in a connected position of the reversible fluidic connection. In some embodiments, wherein the first magnet and the second magnet provides a sealing pressure in a range of 200 kPa to 600 kPa in a connected position of the reversible fluidic connection. In some embodiments, the first magnet is a ring magnet. In some embodiments, the second magnet is a disc magnet. In some embodiments, the device, method, and system disclosed herein comprise a rechargeable power source. In some embodiments, the device, method, and system disclosed herein comprises a user interface configured to allow user selection of at least one function selected from: saliva collection, saliva ejection, saliva rinsing, tooth brushing, bristle retraction, bristle advancement, or bristle disinfection. In some embodiments, the device, method, and system disclosed herein comprises a user interface configured to allow user selection of at least one preprogrammed sequence of functions selected from: saliva rinsing, and then saliva collection; and saliva rinsing, then saliva collection, and then tooth brushing. In some embodiments, the device, method, and system disclosed herein comprise a base, wherein the base is fluidly connected to the device via the reversible fluidic connection. In some embodiments, the base comprises an outlet, wherein the outlet is fluidly connected to the reversible fluidic connection. In some embodiments, the base comprises a power source. In some embodiments, the handheld handle comprises at least one connection interface configurable to connect to the toothbrush, or the suction tip, or the toothbrush and the suction tip.

Also described herein, in various embodiments, are bi-functional portable handheld devices for saliva collection and testing for daily monitoring of physiological conditions of a subject, the device comprising: a handheld handle comprising a saliva collector and ejector, the saliva collector and ejector comprising: a first valve electrically configurable in an open position and a closed position; a suction tip; a saliva reservoir, a bi-directional pump electrically configurable for a withdrawal, an ejection, and a stop; a reversible fluidic connection configurable in a connected position and a disconnected position, wherein the first valve is fluidly connected directly to the saliva reservoir, the suction tip, the bi-directional pump, and the reversible fluidic connection, and wherein the saliva reservoir is fluidly connected directly to the bi-directional pump. In some embodiments, the device, system, and method disclosed herein comprises a toothbrush comprising: a toothbrush head comprising a plurality of bristles, wherein the toothbrush head is configured to retract the plurality of bristles; and a UV light source configured to disinfect the retracted plurality of bristles. In some embodiments, the toothbrush is configured to retract the plurality of bristles mechanically or electrically. In some embodiments, the toothbrush comprises a replaceable platform configured to allow replacement of the toothbrush head. In some embodiments, the toothbrush head is configured to put the plurality of bristles into motion. In further embodiments, the motion of the plurality of bristles comprises one or more selected from: oscillation, vibration, translation, and rectilinear movement. In some embodiments, the devices, methods, and systems disclosed herein comprise a second valve electrically configurable in the open position and the closed position. In further embodiments, the first valve, the second valve, and the reversible fluidic connection are configured to function independently. In further embodiments, an inlet configured to allow rinsing of the saliva collector and ejector, wherein the inlet is electrically controlled by the second valve. In further embodiments, the second valve is fluidly connected directly to the inlet, the saliva reservoir, the bi-directional pump, and the first valve. In further embodiments, the fluidic connection between the inlet and the second valve, the saliva reservoir and the second valve, the first valve and the suction tip, the first valve and the saliva reservoir, the saliva reservoir and the bi-directional pump, the first valve and the second valve, or the first valve and the reversible fluidic connection is via at least one internal tube. In further embodiments, the fluid connection between the inlet and the second valve, the saliva reservoir and the second valve, the first valve and the suction tip, the first valve and the saliva reservoir, the saliva reservoir and the bi-directional pump, the first valve and the second valve, or the first valve and the reversible fluidic connection is bi-directional. In some embodiments, the UV light source is at least one UV LED. In some embodiments, the suction tip comprises a filter. In some embodiments, the suction tip is a telescopic suction tip. In some embodiments, the reversible fluidic connection comprises a first magnet on the device and a second magnet on a base accommodating the device. In some embodiments, the first magnet and the second magnet provides an interfacial force in the range of 1 to 50 Newton in a connected position of the reversible fluidic connection. In some embodiments, wherein the first magnet and the second magnet provides a sealing pressure in a range of 200 kPa to 600 kPa in a connected position of the reversible fluidic connection. In some embodiments, the first magnet is a ring magnet. In some embodiments, the second magnet is a disc magnet. In some embodiments, the device, method, and system disclosed herein comprise a rechargeable power source. In some embodiments, the device, method, and system disclosed herein comprises a user interface configured to allow user selection of at least one function selected from: saliva collection, saliva ejection, saliva rinsing, tooth brushing, bristle retraction, bristle advancement, or bristle disinfection. In some embodiments, the device, method, and system disclosed herein comprises a user interface configured to allow user selection of at least one preprogrammed sequence of functions selected from: saliva rinsing, and then saliva collection; and saliva rinsing, then saliva collection, and then tooth brushing. In some embodiments, the device, method, and system disclosed herein comprise a base, wherein the base is fluidly connected to the device via the reversible fluidic connection. In some embodiments, the base comprises an outlet, wherein the outlet is fluidly connected to the reversible fluidic connection. In some embodiments, the base comprises a power source. In some embodiments, the handheld handle comprises at least one connection interface configurable to connect to the toothbrush, or the suction tip, or the toothbrush and the suction tip.

Discloses herein, in some embodiments, are bi-functional portable handheld devices for tooth brushing, saliva collection, and saliva testing for daily monitoring of physiological conditions of a subject, the device comprising: a toothbrush head comprising a plurality of bristles, wherein the toothbrush head is configured to retract the plurality of bristles; a UV light source configured to disinfect the retracted plurality of bristles; a handheld handle comprising a saliva collector and ejector, the saliva collector and ejector comprising: a first valve electrically configurable in an open position and a closed position; a second valve electrically configurable in an open position and a closed position; a suction tip; a saliva reservoir; a water reservoir; a bi-directional pump electrically configurable for a withdrawal, an ejection, and a stop; a reversible fluidic connection configurable in a connected position and a disconnected position; and a base accommodating the handheld device, the base comprising: a detection unit configurable to connect or disconnect to the reversible fluidic connection; a buffer reservoir configurable to connect or disconnect to the detection unit; a disposable cartridge configurable to connect or disconnect to the detection unit, wherein the first valve is fluidly connected directly to the saliva reservoir, the suction tip, the second valve, and wherein the second valve is fluidly connected directly to the water reservoir, the saliva reservoir, and the reversible fluidic connection, and wherein the saliva reservoir is fluidly connected directly to the bi-directional pump. In some embodiments, the disposable cartridge is configurable to hold at least one reagent therewithin. In some embodiments, the base comprises a wireless system configurable to transmit data to or receive data from at least one external source. In some embodiments, the base comprises a microprocessor. In some embodiments, the toothbrush is configured to retract the plurality of bristles mechanically or electrically. In some embodiments, the toothbrush comprises a replaceable platform configured to allow replacement of the toothbrush head. In some embodiments, the toothbrush head is configured to put the plurality of bristles into motion. In further embodiments, the motion of the plurality of bristles comprises one or more selected from: oscillation, vibration, translation, and rectilinear movement. In some embodiments, the devices, methods, and systems disclosed herein comprise a second valve electrically configurable in the open position and the closed position. In further embodiments, the first valve, the second valve, and the reversible fluidic connection are configured to function independently. In further embodiments, an inlet configured to allow rinsing of the saliva collector and ejector, wherein the inlet is electrically controlled by the second valve. In further embodiments, the second valve is fluidly connected directly to the inlet, the saliva reservoir, the bi-directional pump, and the first valve. In further embodiments, the fluidic connection between the inlet and the second valve, the saliva reservoir and the second valve, the first valve and the suction tip, the first valve and the saliva reservoir, the saliva reservoir and the bi-directional pump, the first valve and the second valve, or the first valve and the reversible fluidic connection is via at least one internal tube. In further embodiments, the fluid connection between the inlet and the second valve, the saliva reservoir and the second valve, the first valve and the suction tip, the first valve and the saliva reservoir, the saliva reservoir and the bi-directional pump, the first valve and the second valve, or the first valve and the reversible fluidic connection is bi-directional. In some embodiments, the UV light source is at least one UV LED. In some embodiments, the suction tip comprises a filter. In some embodiments, the suction tip is a telescopic suction tip. In some embodiments, the reversible fluidic connection comprises a first magnet on the device and a second magnet on a base accommodating the device. In some embodiments, the first magnet and the second magnet provides an interfacial force in the range of 1 to 50 Newton in a connected position of the reversible fluidic connection. In some embodiments, wherein the first magnet and the second magnet provides a sealing pressure in a range of 200 kPa to 600 kPa in a connected position of the reversible fluidic connection. In some embodiments, the first magnet is a ring magnet. In some embodiments, the second magnet is a disc magnet. In some embodiments, the device, method, and system disclosed herein comprise a rechargeable power source. In some embodiments, the device, method, and system disclosed herein comprises a user interface configured to allow user selection of at least one function selected from: saliva collection, saliva ejection, saliva rinsing, tooth brushing, bristle retraction, bristle advancement, or bristle disinfection. In some embodiments, the device, method, and system disclosed herein comprises a user interface configured to allow user selection of at least one preprogrammed sequence of functions selected from: saliva rinsing, and then saliva collection; and saliva rinsing, then saliva collection, and then tooth brushing. In some embodiments, the device, method, and system disclosed herein comprise a base, wherein the base is fluidly connected to the device via the reversible fluidic connection. In some embodiments, the base comprises an outlet, wherein the outlet is fluidly connected to the reversible fluidic connection. In some embodiments, the base comprises a power source. In some embodiments, the handheld handle comprises at least one connection interface configurable to connect to the toothbrush, or the suction tip, or the toothbrush and the suction tip.

Discloses herein, in some embodiments, are bi-functional portable handheld devices for saliva collection and testing for daily monitoring of physiological conditions of a subject, the device comprising: a handheld handle comprising a saliva collector and ejector, the saliva collector and ejector comprising: a first valve electrically configurable in an open position and a closed position; a second valve electrically configurable in an open position and a closed position; a suction tip; a saliva reservoir; a water reservoir; a bi-directional pump electrically configurable for a withdrawal, an ejection, and a stop; a reversible fluidic connection configurable in a connected position and a disconnected position; and a base accommodating the handheld device, the base comprising: a detection unit configurable to connect or disconnect to the reversible fluidic connection; a buffer reservoir configurable to connect or disconnect to the detection unit; a disposable cartridge configurable to connect or disconnect to the detection unit, wherein the first valve is fluidly connected directly to the saliva reservoir, the suction tip, the second valve, and wherein the second valve is fluidly connected directly to the water reservoir, the saliva reservoir, and the reversible fluidic connection, and wherein the saliva reservoir is fluidly connected directly to the bi-directional pump. In some embodiments, the device, system, and method disclosed herein comprises a toothbrush comprising: a toothbrush head comprising a plurality of bristles, wherein the toothbrush head is configured to retract the plurality of bristles; and a UV light source configured to disinfect the retracted plurality of bristles. In some embodiments, the disposable cartridge is configurable to hold at least one reagent therewithin. In some embodiments, the base comprises a wireless system configurable to transmit data to or receive data from at least one external source. In some embodiments, the base comprises a microprocessor. In some embodiments, the toothbrush is configured to retract the plurality of bristles mechanically or electrically. In some embodiments, the toothbrush comprises a replaceable platform configured to allow replacement of the toothbrush head. In some embodiments, the toothbrush head is configured to put the plurality of bristles into motion. In further embodiments, the motion of the plurality of bristles comprises one or more selected from: oscillation, vibration, translation, and rectilinear movement. In some embodiments, the devices, methods, and systems disclosed herein comprise a second valve electrically configurable in the open position and the closed position. In further embodiments, the first valve, the second valve, and the reversible fluidic connection are configured to function independently. In further embodiments, an inlet configured to allow rinsing of the saliva collector and ejector, wherein the inlet is electrically controlled by the second valve. In further embodiments, the second valve is fluidly connected directly to the inlet, the saliva reservoir, the bi-directional pump, and the first valve. In further embodiments, the fluidic connection between the inlet and the second valve, the saliva reservoir and the second valve, the first valve and the suction tip, the first valve and the saliva reservoir, the saliva reservoir and the bi-directional pump, the first valve and the second valve, or the first valve and the reversible fluidic connection is via at least one internal tube. In further embodiments, the fluid connection between the inlet and the second valve, the saliva reservoir and the second valve, the first valve and the suction tip, the first valve and the saliva reservoir, the saliva reservoir and the bi-directional pump, the first valve and the second valve, or the first valve and the reversible fluidic connection is bi-directional. In some embodiments, the UV light source is at least one UV LED. In some embodiments, the suction tip comprises a filter. In some embodiments, the suction tip is a telescopic suction tip. In some embodiments, the reversible fluidic connection comprises a first magnet on the device and a second magnet on a base accommodating the device. In some embodiments, the first magnet and the second magnet provides an interfacial force in the range of 1 to 50 Newton in a connected position of the reversible fluidic connection. In some embodiments, wherein the first magnet and the second magnet provides a sealing pressure in a range of 200 kPa to 600 kPa in a connected position of the reversible fluidic connection. In some embodiments, the first magnet is a ring magnet. In some embodiments, the second magnet is a disc magnet. In some embodiments, the device, method, and system disclosed herein comprise a rechargeable power source. In some embodiments, the device, method, and system disclosed herein comprises a user interface configured to allow user selection of at least one function selected from: saliva collection, saliva ejection, saliva rinsing, tooth brushing, bristle retraction, bristle advancement, or bristle disinfection. In some embodiments, the device, method, and system disclosed herein comprises a user interface configured to allow user selection of at least one preprogrammed sequence of functions selected from: saliva rinsing, and then saliva collection; and saliva rinsing, then saliva collection, and then tooth brushing. In some embodiments, the device, method, and system disclosed herein comprise a base, wherein the base is fluidly connected to the device via the reversible fluidic connection. In some embodiments, the base comprises an outlet, wherein the outlet is fluidly connected to the reversible fluidic connection. In some embodiments, the base comprises a power source. In some embodiments, the handheld handle comprises at least one connection interface configurable to connect to the toothbrush, or the suction tip, or the toothbrush and the suction tip.

Also described herein, in various embodiments, are methods of saliva collection and testing using a portable handheld device for daily monitoring of physiological conditions of a subject, the method comprising: rinsing the device using a bi-directional pump using liquid from at least one selected from a saliva reservoir, a water reservoir, a suction tip, and a base accommodating the device, wherein a first valve and a second valve are in an open position and a reversible fluidic connection is in a connected position; collecting saliva from the suction tip to the saliva reservoir, wherein the first valve is in the open position; and transmitting the saliva from the saliva reservoir to a detection unit enclosed in the base, wherein the second valve is in the open position and the reversible fluidic connection is in the connected position; testing the saliva for at least one biomarker in the detection unit; wherein the first valve is fluidly connected directly to the saliva reservoir, the suction tip, the second valve, and wherein the second valve is fluidly connected directly to the water reservoir, the saliva reservoir, and the reversible fluidic connection, and wherein the saliva reservoir is fluidly connected directly to the bi-directional pump, and the reversible fluidic connection is fluidly connected to the detection unit. In some embodiments, the toothbrush is configured to retract the plurality of bristles mechanically or electrically. In some embodiments, the toothbrush comprises a replaceable platform configured to allow replacement of the toothbrush head. In some embodiments, the toothbrush head is configured to put the plurality of bristles into motion. In further embodiments, the motion of the plurality of bristles comprises one or more selected from: oscillation, vibration, translation, and rectilinear movement. In some embodiments, the devices, methods, and systems disclosed herein comprise a second valve electrically configurable in the open position and the closed position. In further embodiments, the first valve, the second valve, and the reversible fluidic connection are configured to function independently. In further embodiments, an inlet configured to allow rinsing of the saliva collector and ejector, wherein the inlet is electrically controlled by the second valve. In further embodiments, the second valve is fluidly connected directly to the inlet, the saliva reservoir, the bi-directional pump, and the first valve. In further embodiments, the fluidic connection between the inlet and the second valve, the saliva reservoir and the second valve, the first valve and the suction tip, the first valve and the saliva reservoir, the saliva reservoir and the bi-directional pump, the first valve and the second valve, or the first valve and the reversible fluidic connection is via at least one internal tube. In further embodiments, the fluid connection between the inlet and the second valve, the saliva reservoir and the second valve, the first valve and the suction tip, the first valve and the saliva reservoir, the saliva reservoir and the bi-directional pump, the first valve and the second valve, or the first valve and the reversible fluidic connection is bi-directional. In some embodiments, the UV light source is at least one UV LED. In some embodiments, the suction tip comprises a filter. In some embodiments, the suction tip is a telescopic suction tip. In some embodiments, the reversible fluidic connection comprises a first magnet on the device and a second magnet on a base accommodating the device. In some embodiments, the first magnet and the second magnet provides an interfacial force in the range of 1 to 50 Newton in a connected position of the reversible fluidic connection. In some embodiments, wherein the first magnet and the second magnet provides a sealing pressure in a range of 200 kPa to 600 kPa in a connected position of the reversible fluidic connection. In some embodiments, the first magnet is a ring magnet. In some embodiments, the second magnet is a disc magnet. In some embodiments, the devices, methods, and systems disclosed herein comprise a rechargeable power source. In some embodiments, the device, method, and system disclosed herein comprises a user interface configured to allow user selection of at least one function selected from: saliva collection, saliva ejection, saliva rinsing, tooth brushing, bristle retraction, bristle advancement, or bristle disinfection. In some embodiments, the device, method, and system disclosed herein comprises a user interface configured to allow user selection of at least one preprogrammed sequence of functions selected from: saliva rinsing, and then saliva collection; and saliva rinsing, then saliva collection, and then tooth brushing. In some embodiments, the device, method, and system disclosed herein comprise a base, wherein the base is fluidly connected to the device via the reversible fluidic connection. In some embodiments, the base comprises an outlet, wherein the outlet is fluidly connected to the reversible fluidic connection. In some embodiments, the base comprises a power source. In some embodiments, the handheld handle comprises at least one connection interface configurable to connect to the toothbrush, or the suction tip, or the toothbrush and the suction tip. In some embodiments, the methods include a disposable cartridge configurable to hold at least one reagent therewithin. In some embodiments, the base comprises a wireless system configurable to transmit data to or receive data from at least one external source. In some embodiments, the base comprises a microprocessor. In some embodiments, the methods comprise withdrawing liquid from the suction tip, the base, or the suction tip and a base to at least one of the water reservoir and the saliva reservoir. In some embodiments, said liquid is tap water or a saliva rinsing solution. In some embodiments, the base comprises a buffer reservoir configurable to fluidly connect to the detection unit; and a disposable cartridge configurable to fluidly connect to the detection unit.

### Overview

In some embodiments, the systems, devices, and methods disclosed herein are configurable for tooth brushing, saliva collection, and saliva testing for salivary biomarker testing with minimal or negligible interferences to each individual function or step within a single portable handheld device. In some embodiments, the systems, devices, and methods for saliva collection and testing is conjugated to a daily routine, i.e. tooth brushing, so that it is easy and convenient for the user to perform saliva collection and testing every time when he or she brushes the teeth. In some embodiments, the systems, devices, and methods disclosed herein also separate tooth brushing elements from saliva collection and testing elements of the device to reduce possible interference or invalidation of saliva testing by saliva residual from previous collections, toothpaste residual, or other contamination sources. To ensure accurate and reliable saliva collection and testing, in some embodiments, the saliva rinsing function or step is required until contamination from previous usages is reduced below a pre-determined threshold. Further, in some cases, saliva collection and testing is only activated after a sensing of contamination meets a pre-determined threshold. In some embodiments, the saliva suction tip has a separate and independent connection interface to the rest of the saliva collector and ejector and this connection interface is not interfered by the toothbrush head, tooth paste, or other possible external sources. In some embodiments, the handheld device allows user selection of functions in the exact order that they are included in preprogrammed sequences to ensure proper withdrawal of saliva and minimal contamination by tooth brushing. In some embodiments, the systems, devices, and methods disclosed herein use active bi-directional pumping but not any capillary forces. Active pumping is bi-directional and it ensures effective cleansing and reliable repetitive usages. In some embodiments, the systems, devices, and methods disclosed herein are configurable to retract toothbrush bristles for sterilization using enclosed UV light LEDs. The retraction and sterilization of bristles reduces contamination of the toothbrush and possible connection of the toothbrush to the saliva collector and tester, thus ensures accurate and reliable saliva collection and testing. In some embodiments, the collected saliva is transported to a detection unit at the base for testing in order to reduce possible interferences to the testing procedure by external sources such as the toothbrush, the bristles, the tooth paste, water, or saliva rising solutions. In some embodiments, a saliva reservoir is used to store collected saliva for testing at a selected time point after the collection. In some embodiments, the saliva reservoir is electrically controlled by a switch, a valve, a fitting, or a reversible connection so that it can be hermetically sealed from contamination or deterioration sources. In some embodiments, the detection unit for actual saliva testing is located in the base. The base is more stable and less exposed to movement, motion, or other accidental causes comparing with the handheld handle, therefore, the biomarker testing unit, signal processing unit, such as the sensors, electrodes, circuits, and other elements are better protected from possible interferences and damages from tooth brushing motions and other accidental stimulus. Thus, the testing at the base is safer, more accurate, and more reliable. In addition, the handheld handle is lighter in weight and convenient to use with the detection unit and the associated elements located in the base.

In some embodiments, the saliva reservoir provides a temporary storage of freshly collected saliva sample so that the sample is delivered to the base for testing of salivary biomarker at a later point. The saliva reservoir advantageously ensure that the saliva sample volume is sufficient for a valid biomarker testing before the sample is delivered to the detection unit, so that it reduces the number of invalid sample transportation or testing due to insufficient sampling. It also allows accurate and reliable sampling since the saliva sample is only stored for a very short period of time before the salivary biomarker starts to degrade to an extent that might influence a testing result. In some embodiments, the freshly collected saliva samples are tested instantly in order to minimize changes that may occur in saliva or salivary biomarkers over time. Such instant testing of saliva preserves salivary biomarkers and allows sensitive detection and monitoring of different biomarkers. In some embodiments, saliva rinsing is merely at the handheld handle so that fluidic and electrical elements in the base are not exposed to damages that water or rinsing solution might cause. In some embodiments, saliva transportation is merely controlled by the device but not by the user. Specifically, saliva transportation is controlled by the base, and only when the testing elements such as the buffer, detection unit, reagent, are in a ready and functional condition, saliva transportation can be enabled. Further, activation of saliva transportation function or step also requires a successful saliva collection, i.e., uncontaminated fresh saliva sample with a sufficient volume, at the handle so that a proper testing can be carried out.

### Saliva Rinsing

In some embodiments, the systems, devices, and methods disclosed herein include a saliva rinsing function or step or the like. In some embodiments, saliva rinsing is a prerequisite step/function before any saliva is collected for biomarker testing. In some embodiments, saliva rinsing is an essential step/function to ensure accurate and reliable saliva collection for testing of biomarkers. In some embodiments, saliva rinsing is performed after detection of an unsatisfactory level of saliva residual or other sources of contamination in the device. In some embodiments, saliva rinsing is automatically activated after detection of contamination above a pre-selected threshold. In some embodiments, saliva rinsing is performed to eliminate saliva residual from previous collection or other sources of contamination. In some embodiments, saliva rinsing rinses one or more elements selected from: the inlet, the first valve, the second valve, the third valve, an internal tube, a suction tip, a filter, a saliva reservoir, a reversible fluidic connection, a tooth brush head, bristles, a base accommodating the hand-held device, a syringe, a piston, and a pump, a tube, and a handheld handle. In some embodiments, the saliva rinsing is by active means. In further embodiments, the saliva rinsing uses a bi-directional pump, or a syringe. In some embodiments, the saliva rinsing uses water force/pressure from tap water. In some embodiments, the saliva rinsing lasts for a pre-determined period of time. In some embodiments, the saliva rinsing lasts until the saliva residual or other sources of contamination has been reduced below a pre-determined threshold. In some embodiments, saliva rinsing uses tap water or chemical solution. In some embodiments, the rinsing solution is introduced to the handheld device from one or more selected from the inlet, the suction tip, the base, and the reversible fluidic connection. In some embodiments, the rinsing solution exits the handheld device from one or more selected from the inlet, the suction tip, the base, and the reversible fluidic connection. In some embodiments, the saliva rinsing step requires soaking the handheld device in water or rinsing solution. In some embodiments, the rinsing step is to break residual proteins and/or molecules, eliminate bacteria, microorganism, and/or toothpaste, and other possible contamination sources of saliva biomarkers. In some embodiments, at least one selected from: the first valve, the reversible fluidic connection, and the second valve, the third valve, the saliva reservoir is in an open or connected position. In some embodiments, the rinsing solution travels through all the individual elements of a saliva collector and ejector during rinsing and/or rinsing solution withdrawal to ensure effective rinsing of the device.

In some embodiments, a proper saliva rinsing step or function only rinses the handheld handle of the device. In some embodiment a proper saliva rinsing step or function does not rinse the base and elements and connections enclosed therewithin. In some embodiments, the base and elements and connections enclosed therewithin is rinsed separately from a saliva rinsing step or function. In some cases, the base and elements and connections enclosed therewithin are rinsed using at least a buffer solution stored within the base. In some embodiments, saliva rinsing is only activated when the handheld handle is properly detached from the base. In some embodiments, saliva rinsing is disabled when the handheld handle is properly placed back in the base. In some embodiments, saliva rinsing is disabled when the handheld handle is fluidly connected to the base. In some embodiments, saliva rinsing is disabled by electrically closing a valve or turning off a switch so that the water reservoir is fluidly disconnected to the reversible fluidic connection or the base. In some embodiments, the base and elements and connections enclosed therewithin is always disconnected fluidly from the water reservoir. In some embodiments, the base and elements and connections enclosed therewithin is always protected from exposure to water or saliva rinsing solution from the handheld handle.

### Saliva Collection

In some embodiments, the systems, devices, and methods disclosed herein include a saliva collection function, step, element, or the like. In some embodiments, saliva is collected from a subject via a sufficient contact of the subject's saliva with a suction tip. In some embodiments, saliva is pumped by a bi-directional pump from the suction tip into a saliva reservoir through at least one valve and/or a reversible fluidic connection. In some embodiments, the saliva is pumped by a bi-directional pump from the suction tip into the base through at least one valve and/or a reversible fluidic connection, when the handheld device is properly placed on the base and the reversible fluidic connection is connected. In some embodiments, the saliva is pumped to the saliva reservoir and later from the saliva reservoir to the base accommodating the device after the handheld device is properly placed on the base and the reversible fluidic connection is in a connected position.

In some embodiments, the saliva collection lasts for a pre-determined period of time. In some embodiments, the saliva collection lasts until the collected saliva volume reaches a pre-determined threshold. In some embodiments, the duration of saliva collection is automatically controlled by the device. In other embodiments, the duration of saliva collection is user-controlled. In some embodiments, a sound, visual, or a motion signal is given by the device automatically when a successful saliva collection has been achieved. In some embodiments, a sound, visual, or a motion signal is given by the device automatically when a saliva collection is not successful. In some embodiments, a saliva collection of about 0.1 ml to about 10 ml is required for each successful saliva collection step or function. In some embodiments, a successful saliva collection is performed in less than 1 second to less than 10 minutes. In some embodiments, the collected saliva is transported automatically from the saliva reservoir to the base when the handheld device is properly positioned to the base accommodating the device. In some embodiments, the saliva transportation step is not performed when the tooth brushing function is active to minimize possible interference between functions.

### Saliva Ejection

In some embodiments, the saliva is pumped by at least one bi-directional pump out of the saliva reservoir to the base accommodating the handheld device for testing sequentially through at least one valve, at least one reversible fluidic connection, or at least one valve and one reversible connection. In some embodiments, the collected or tested saliva is pumped by at least one bi-directional pump from the reservoir to the suction tip through at least one valve, at least one reversible fluidic connection, or at least one valve and one reversible connection. In some embodiments, the collected or tested saliva is pumped by at least one bi-directional pump from the reservoir to an inlet/outlet through at least one valve, at least one reversible fluidic connection, or at least one valve and one reversible connection. In some embodiments, the collected or tested saliva is pumped by at least one bi-directional pump from the saliva reservoir to at least one selected from: an inlet/outlet, a suction tip, and a base. In some embodiments, the collected or tested saliva is pumped by at least one bi-directional pump from the base to at least one selected from: an inlet/outlet, a suction tip, an outlet at the base, and a saliva reservoir. In some embodiments, the collected or tested saliva is pumped from the base to the suction tip for disposal through at least one valve, at least one reversible fluidic connection, or at least one valve and one reversible connection. In some embodiments, the collected or tested saliva is pumped from the base to an inlet/outlet for disposal through at least one valve, at least one reversible fluidic connection, or at least one valve and one reversible connection. In some embodiments, the collected or tested saliva is disposed from the base directly through an outlet. In some embodiments, the outlet/inlet is equivalent to a dispenser.

### Saliva Transportation

In some embodiments, saliva is transported form the saliva reservoir to the base through a valve and a reversible fluidic connection. In some embodiments, saliva is transported form the saliva reservoir to the base when the valve is in a position that it only allows connection between the saliva reservoir and the reversible fluidic connection and the reversible fluidic connection is in a connected position to the base. In some embodiments, saliva is transported form the saliva reservoir to the base automatically when the handheld handle is properly placed back or attached to the base. In some embodiments, the saliva is transported before a tooth-brushing function can be activated by the device. In some embodiments, saliva transportation is a prerequisite step before a tooth brushing step is activated. In some embodiments, saliva transportation can be activated only after a saliva collection of a predetermined volume in a predetermined time window is achieved. In some embodiments, saliva transportation can be activated only after a predetermined volume of liquid is detected in the saliva reservoir. In some embodiments, the predetermined volume is no less than 1 milliliter. In some embodiments, the predetermined time window is in the range of no less than 0.1 second to no more than 10 minutes. In some embodiments, the predetermined volume is in the range of no less than 10 microliter to no less than 10 milliliter. In some embodiments, an indication is given when the saliva is not transported from the saliva reservoir in a predetermined period of time after a successful saliva collection. In some embodiments, the predetermined period of time is no more than 1 second to no more than 10 minutes. In some embodiments, an indication is generated when the handheld handle is not properly placed back in the base. In some embodiments, said indication is a vibration, a sound, a light, a display, or a combination of the like. In some embodiments, saliva collection and saliva transportation steps are performed together when the handheld handle is fluidly connected to the base. In some embodiments, saliva transportation is automatically controlled by the system but not controllable by a user selection. In some embodiments, saliva transportation is activated when the handheld handle is properly placed back in the base, and the fluidic connection between the handle and the base has been established. In some embodiments, saliva transportation is activated when the handheld handle is properly placed back in the base, and at least one predetermined condition has been met. In some embodiments, the fluidic connection between the handle and the base is established when at least one predetermined condition has been met. In some embodiments, a predetermined condition is at least one selected from: a saliva rinsing and a saliva collection step are performed successfully, a liquid volume within the saliva reservoir is above a predetermined threshold, a contamination sensing meets a predetermined standard, the buffer volume is above a threshold, the detection unit is ready for testing a new saliva sample, the reagent level is above a threshold, the reversible fluidic connection in the handle and the reversible fluidic connection in the base are in a connected position, at least one sensor is functioning normal, the microprocessor is functioning normal, and the pump at the base is functioning normal.

### Internal Connections

In some embodiments, connection between any two elements selected from the list are through internal tubing: a first bi-directional pump, a second bi-directional pump, a saliva reservoir, a first valve, a second valve, a third valve, a suction tip, a base, an inlet/outlet, and a syringe. In some embodiments, the fluidic path via tubing between any two elements selected from abovementioned list is hermetically sealed when the connection between the two elements is properly eliminated. In some embodiments, all the internal tubes are bi-directional so that fluidic communication in two directions is not limited or restricted by the tubing. In some embodiments, the tubes do not exert any dynamic or active force on the fluid flows therewithin. In some embodiments, the tube has a cross-sectional diameter in the range of 0.01 mm to 8 mm.

In some embodiments, the suction tip is fluidly connected to the saliva reservoir through at least one valve, at least one reversible fluidic connection, or at least one valve and one reversible connection. In some embodiments, the bi-directional pump is fluidly connected to the saliva reservoir via at least one internal tube but without a valve or a reversible fluidic connection. In other embodiments, the bi-directional pump is fluidly connected to the saliva reservoir through at least one valve, at least one reversible fluidic connection, or at least one valve and one reversible connection. In some embodiments, the suction tip is fluidly connected to the bi-directional pump through at least one valve, at least one reversible fluidic connection, or at least one valve and one reversible connection. In some embodiments, an inlet/outlet is fluidly connected to the bi-directional pump through at least one valve, at least one reversible fluidic connection, or at least one valve and one reversible connection. In some embodiments, an inlet/outlet is fluidly connected to a reservoir through at least one valve, at least one reversible fluidic connection, or at least one valve and one reversible connection. In some embodiments, an inlet/outlet is fluidly connected to the suction tip through at least one valve, at least one reversible fluidic connection, or at least one valve and one reversible connection. In some embodiments, an inlet/outlet is fluidly connected to a base through at least one valve, at least one reversible fluidic connection, or at least one valve and one reversible connection. In some embodiments, a base is fluidly connected to a suction tip through at least one valve, at least one reversible fluidic connection, or at least one valve and one reversible connection. In some embodiments, a base is fluidly connected to a reservoir through at least one valve, at least one reversible fluidic connection, or at least one valve and one reversible connection. In some embodiments, a base is fluidly connected to a bi-direction pump through at least one valve, at least one reversible fluidic connection, or at least one valve and one reversible connection. In some embodiments, the internal connections of the handheld device is hermetically sealed when all the valves, the reversible fluidic connections, and switches are properly closed or disconnected. In some embodiments, tubing and tube are interchangeable and are equivalent elements in the systems, devices, and methods disclosed herein.

In some embodiments, the internal tube includes at least one selected from: a two-way fitting, a three way fitting, a four way fitting, a tube fitting, and a tube adaptor. In some embodiments, the internal tubes are made of materials that are chemically inert to saliva, water, saliva rinsing solution, and saliva testing chemicals.

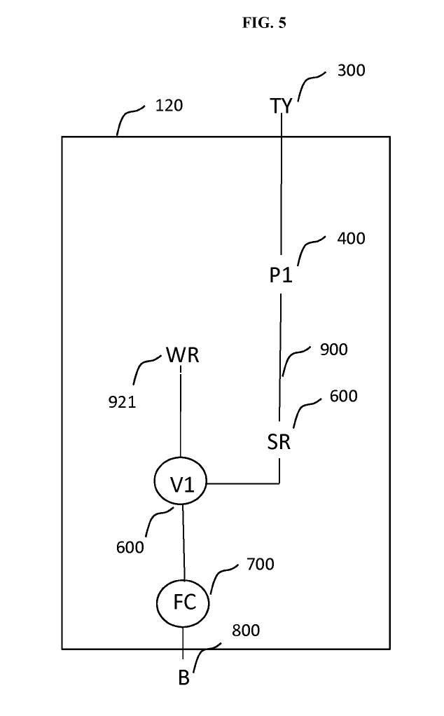

Referring to FIG. 5, in a particular embodiment, the handheld portable device 10 for saliva collection includes a handheld handle 120 and a base 800 accommodating the handheld device for fluidic, electrical, and mechanical connections. In the same embodiment, the handle 120 is fluidly connectable to a suction tip 300 in a removable manner via at least one connection interface. In some embodiments, the connection interface to an optional toothbrush head and the suction tip is the same. In other embodiments, the connection interface to the optional toothbrush head and the suction tip is different and independent. In this embodiment, the suction tip 300 is fluidly connected directly to a bi-directional pump 400. In some embodiments, the pump 500 is a peristaltic pump. In the same embodiment, the pump 400 is directly connected to a saliva reservoir 600. In this embodiment, a valve 500 is fluidly connected to the saliva reservoir 600, a reversible fluidic connection 700, and a water reservoir 920. In some embodiments, the water reservoir 920 is located higher than the saliva reservoir 600 to facilitate liquid flow into the saliva reservoir 600 for rinsing purposes. In this particular embodiment, the valve 500 is electrically controlled to allow fluid communication between any two or all three elements that are fluidly connected to the valve. In some embodiments, these three elements are the saliva reservoir 600, the reversible fluidic connection 700, and the water reservoir 920. In this embodiment, the reversible fluidic connection 700 directly connects to the base 800. The fluidic connections between any two above-mentioned elements are via internal tubing 900.

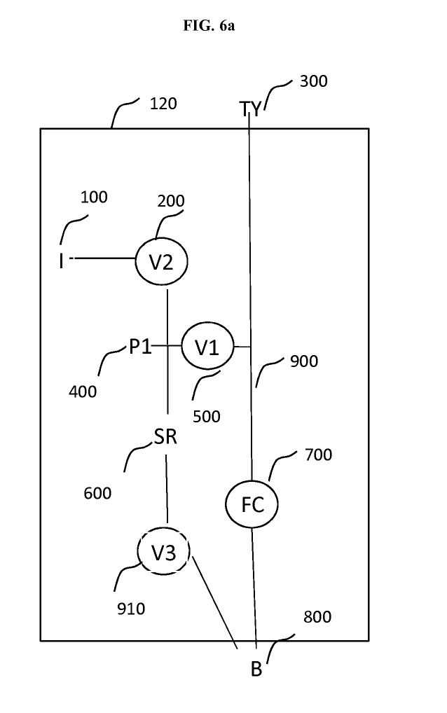

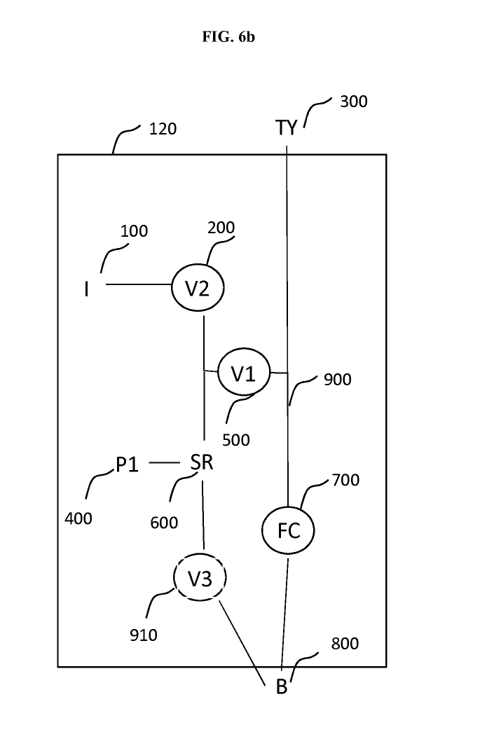

Referring to FIG. 6a, in a particular embodiment, the handheld portable device encloses internal connections within the handheld handle 120. In this embodiment, a suction tip 300 is fluidly connected to a first valve 500. The first valve 500 is then directly connected to a first pump 400 and a saliva reservoir 600, and a reversible fluidic connection 700, and optionally to a second valve 200. In the same embodiment, the reversible fluidic connection 700 directly connects to the base 800. The optional second valve 200 is connected to an inlet 100, the pump 400, and the saliva reservoir 600. In this particular embodiment, an optional third valve 910 is directly connected to the saliva reservoir 600 and the base 800. The fluidic connections between any two above-mentioned elements are via internal tubing 900.

Referring to FIG. 6b, in a particular embodiment, the handheld portable device encloses internal connections within the handheld handle 120. In this embodiment, a suction tip 300 is fluidly connected to a first valve 500. The first valve 500 is then directly connected to a saliva reservoir 600, and a reversible fluidic connection 700, and optionally to a second valve 200. In the same embodiment, the reversible fluidic connection 700 directly connects to the base 800. The optional second valve 200 is connected to an inlet 100, and the saliva reservoir 600. In this particular embodiment, an optional third valve 910 is directly connected to the saliva reservoir 600 and the base 800. The saliva reservoir 600 is directly connected to a first pump 400. The fluidic connections between any two above-mentioned elements are via internal tubing 900.

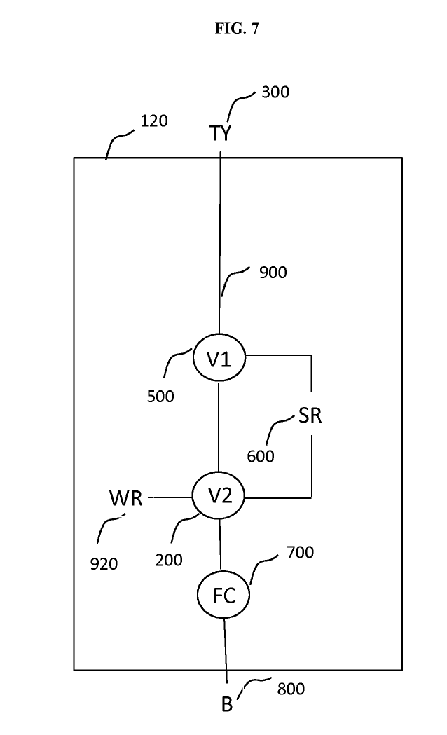

Referring to FIG. 7, in a particular embodiment, the handheld portable device encloses internal connections within the handheld handle 120. In this embodiment, a suction tip 300 is fluidly connected to a first valve 500. The first valve 500 is then directly connected to a saliva reservoir 600, and a second valve 200. The second valve 200 is connected directly to the saliva reservoir 600, a water reservoir 920, and a reversible fluidic connection 700. In the same embodiment, the reversible fluidic connection 700 directly connects to the base 800. The saliva reservoir 600 is directly connected to a first pump and the water reservoir is directly connected to the first pump or a second pump. The fluidic connections between any two above-mentioned elements are via internal tubing 900.

### Valves

In some embodiments, the systems, devices, and methods disclosed herein include a valve or the like. In certain cases, the valve has a size suitable to connect the internal tubing within the handheld device. In some embodiments, the valve has a cross-section diameter of no less than 0.5 mm and no greater than 5 mm. In some embodiments, the valve is a micro-valve or a mini-valve. In some embodiments, the valve is electrically controlled to be in an open or a closed position. In some embodiments, at least one valve fluidly connects two selected from: a first bi-directional pump, a second bidirectional pump, a saliva reservoir, a first valve, a second valve, a suction tip, a base, an inlet, a syringe. In some embodiments, at least one valve creates fluidly connection between the saliva reservoir and the suction tip. In some embodiments, at least one valve creates fluidly connection between the saliva reservoir and the base. In some embodiments, at least one valve creates fluidly connection between the base and the suction tip. In some embodiments, the valve creates a two way fluid connection. In other embodiments, the valve creates a three way fluid connection. In further embodiments, any two or all three elements linked to a three way valve are fluidly connected by selection of different connecting modes at the valve. In other embodiments, the valve creates a four way fluid connection. In further embodiments, any two, three, or all four elements linked to a four way valve are fluidly connected by selection of different connecting modes at the valve. In some embodiments, a valve is interchangeable with an electrically controlled connector, switch, or fitting. In some embodiments, a valve is equivalent to an electrically controlled connector, switch, or fitting.

In some embodiments, the valve disclosed herein is a three-way valve configurable to allow bi-directional fluidic communication between any two or any three pathways linked at the three-way valve. In some embodiments, the actual direction of fluid flow is determined by the bi-directional pump with or without the assistance of fluid gravity or fluid potential energy. In some embodiments, during saliva rising, the valve is configured to allow fluidic communication from the water reservoir to the reversible fluidic connection and to the saliva reservoir. In some embodiments, during saliva collection, the valve is configured to eliminate fluidic communication between the saliva reservoir and the reversible fluidic connection and between the saliva reservoir and the water reservoir. In some embodiments, during saliva collection, the valve is configured to allow fluidic communication between the saliva reservoir and the reversible fluidic connection, eliminate fluidic communication between the saliva reservoir and the water reservoir and eliminate fluidic communication between the water reservoir and the reversible fluidic connection. In some embodiments, during saliva transportation, the valve is configured to allow fluidic communication from the saliva reservoir to the reversible fluidic connection and eliminate fluidic communication between the water reservoir and the reversible fluidic connection and eliminate fluidic communication between the water reservoir and the saliva reservoir.

In some embodiments, the valve disclosed herein is a four-way valve configurable to allow bi-directional fluidic communication between any two, three, or four pathways linked at the valve. In some embodiments, the valve disclosed herein is a five-way valve configurable to allow bi-directional fluidic communication between any two, three, four, or five pathways linked at the valve. In some embodiments, the valve disclosed herein is a six-way valve configurable to allow bi-directional fluidic communication between any two, three, four, five, or six pathways linked at the valve. In some embodiments, the valve disclosed herein is a seven-way valve configurable to allow bi-directional fluidic communication between any two, three, four, five, six, or seven pathways linked at the valve.

### Suction Tips

In some embodiments, the systems, devices, and methods disclosed herein include a suction tip or the like. In some embodiments, the suction tip includes a filter to filter out unwanted particles or substances in the saliva. In some embodiments, the filter is for multiple usages. In some embodiments, the filter is replaceable. In some embodiments, the suction tip and the filter are replaceable. In some embodiment, the suction tip is reversibly connected to the handheld device so that it is changeable after a certain number of usages. In some embodiment, the suction tip is a telescopic suction tip. In some embodiments, the suction tip is actuated by the bi-directional pump for withdrawal or ejection of liquid. In some embodiments, the suction tip can be electrically controlled at a user interface.

In some embodiments, the suction tip has an elongate portion and a tip portion. The tip portion is mechanically and fluidly connected to the elongate portion and then to the rest of the saliva collector and saliva ejector. In some embodiments, the elongate portion is telescopic. In some embodiments, the tip portion makes direct contact with saliva or liquid. In some embodiments, the tip portion has a shape that increases the contacting surface of the suction tip and the saliva. In some embodiments, the tip portion has a bulbous shape. In some embodiments, the tip portion has cylindrical shape. In some embodiments, the tip portion has an elliptical shape, a sphere shape, a flat surface, a concave surface, or a convex surface.

In some embodiments, the suction tip consists of concentric tubular sections designed to slide into one another. In some embodiments, the tip has a direct connection to a bi-directional pump or a syringe within the handheld device. In some embodiments, the suction tip has a length of about 1 cm to 30 cm when the elongate portion is at its maximal length.

### Pumps

In some embodiments, the systems, devices, and methods disclosed herein include at least one bi-directional pump, a bi-directional syringe, or use of the same. In some embodiments, the pump and syringe are interchangeable and equivalent. In some embodiments, the bidirectional pump is directly connected to a valve and/or a saliva reservoir. In some embodiments, the bidirectional pump located in the base and is directly connected to reversible fluidic connection. In certain case, the bi-directional pump pumps saliva, water, rinsing solutions, or other liquid from external sources into the handheld device. In other cases, the bi-directional pump pumps saliva, water, rinsing solutions, or other liquid from within the hand-held device to external sources. In some embodiments, the pump is actuated to pump, suck, withdraw, or eject electrically. In some embodiments, the pump is configured to be turned on and off electrically. In some cases, the pump provides a flow rate of 0 to 80 liter per minute (1 pm). In certain cases, the pump is driven by an electric motor. In some embodiments, the pump is controlled via user selection at a user interface. In some embodiments, the pump is configured to work at different pumping or sucking efficiency or at different flow rate. In further embodiments, the flow rate is preprogrammed or user-selected at a user interface. In some embodiments, the bi-directional pump is configurable to direct liquid flow in two opposite directions. In some embodiments, the bi-directional pump is a pumping element that is configurable to direct liquid flow in two opposite directions. In some embodiments, the pumping direction of the bi-directional pump controls the flow direction of the fluid within the internal connections, the water reservoir, the saliva reservoir, the reversible fluidic connection, the suction tip, the tube, the valve, the switch, the tube fitting, and the base. In some embodiments, the fluid flow direction is controlled by a combination of the pumping direction, and the water gravity and potential energy. In some embodiments, the pumping force is sufficient to overcome the force caused by water gravity and water potential energy. In some embodiments, the bi-directional pump is a peristaltic pump. In some embodiments, the bi-directional pump includes an element for excluding air or vacuum to minimize resistance in pumping within the internal connections and the all the other elements within a handheld handle.

### Saliva Reservoirs

In some embodiments, the systems, devices, and methods disclosed herein include a saliva reservoir or the like. In some embodiments, the saliva reservoir is a container with at least one direct or indirect fluidic connection to one or more selected from a first valve, a bi-directional pump, a second valve, a reversible fluidic connection, a suction tip, a base, an inlet, and a syringe. In some embodiments, the direct fluidic connection between two elements is through internal tubing and without any other additional elements in between the two directly connected elements. In some embodiments, the indirect fluidic connection between two elements is through internal tubing and at least one other additional element in between the two directly connected elements, such as a valve, a switch, a tube fitting, or a connector. In some embodiments, the saliva reservoir is electrically or mechanically controlled by at least one of a switch, a valve, and a reversible connection. In further embodiments, the saliva reservoir is individually controlled by the switch, the valve or the reversible connection to be in an open or a closed position.

In some embodiments, the saliva reservoir is sized to enclose a saliva sample volume sufficient for at least one biomarker testing. In some embodiments, the saliva reservoir has a size range of about 0.1 ml to about 10 ml. In some embodiments, the saliva reservoir includes a mechanical or electrical volume indicator so that the enclosed volume of liquid in the saliva reservoir is visible to a user. In some embodiments, the saliva reservoir has a transparent wall. In some embodiments, the saliva reservoir is made of materials that are chemically inert to saliva or saliva rinsing solutions. In some embodiments, the saliva reservoir is sized to be in the range of about 1 mm³ to about 6 cm³. In some embodiments, the saliva reservoir includes an outlet. In some embodiments, the outlet is electrically controlled by a valve or mechanically controlled by a switch.

In some embodiments, the saliva reservoir includes a sensor for sensing one or more of saliva residuals, tooth paste residuals, proteins, bacteria, molecules, and microorganisms. In some embodiments, if the sensor valve does not meet a pre-determined threshold, a saliva rinsing function is automatically activated until the sensor valve meets the pre-determined threshold. In some embodiments, if the sensor valve does not meet a pre-determined threshold, a saliva collection function is automatically disabled. In some embodiments, the saliva reservoir is replaceable.

### Water Reservoirs

In some embodiments, the water reservoir includes an external filling lid. In some cases, the external filling lid is located so that the opening or closing of the lid is not obstructed by any part of the base when the handheld device is disconnected or properly placed back to the base accommodating the device. In some embodiments, the opening or closing of the external filling lid does not interfere with the detachment or attachment of the handheld handle to the base. In some embodiments, the external filling lid is water tight, air tight, or water tight and air tight when it is properly closed. In some embodiments, the water reservoir is hermetically sealed when the external filling lid is closed and the valve is closed to the water reservoir. In some embodiments, the external filling lid is mechanically controlled by a user. In some cases, the external filling lid is electrically controlled by the system. In some embodiments, the external filling lid automatically closes when the liquid level is above a pre-determined threshold. In some cases, an indication is given when the liquid level is above or below a pre-determined threshold. In some embodiments, said indication is a vibration, a sound, a light, a display, or a combination of the like.

In some embodiments, the water reservoir encloses water and/or saliva rinsing solution for rinsing the internal connections of the handheld handle. In some embodiments, a water reservoir is sized such that a filled water reservoir allows successful saliva rinsing for at least twice to at least ten times before any refill of the reservoir. In some cases, successful saliva rinsing ensures that contamination sources within the internal fluidic pathways or elements are below a pre-determined level. In some cases, successful saliva rinsing ensures the saliva collection step finishes stops in no more than 1 second to no more than 10 minutes. In some embodiments, saliva rinsing rinses at least one selected from: a bi-directional pump, a tube, a tube fitting, a valve, a suction tip, a filter, a reversible fluidic connection, a base, a syringe, a cartridge, a detection unit, an inlet/outlet, a switch, and a saliva reservoir. In some embodiments, the water reservoir is vertically located higher than the saliva reservoir in order to facilitate the saliva rinsing.

In some embodiments, the systems, devices, and methods disclosed herein include a water reservoir or the like. In some embodiments, the water reservoir is enclosed in the handheld handle of the device. In some embodiments, the water reservoir is directly in fluid communication with a valve. In some embodiments, the water reservoir is in direct or indirect fluid communication with a valve, a reversible fluidic connection, a suction tip, a saliva reservoir. In some embodiments, the water reservoir is in indirect fluid communication with a valve, a reversible fluidic connection, a suction tip, a saliva reservoir through at least one of a valve, a switch, a tube fitting, and a reversible fluidic connection. In some embodiments, the water reservoir includes a pumping element so that liquid therewithin is pumped in or out of the water reservoir. In some embodiments, the water reservoir is made of material that is chemically inert to water, saliva, and saliva rinsing solution. In some embodiments, the water reservoir is connected to a bi-directional pump.

In some embodiments, the water reservoir is electrically or mechanically controlled by at least one of a switch, a valve, and a reversible connection. In further embodiments, the water reservoir is individually controlled by the switch, the valve or the reversible connection to be in an open or a closed position.

In some embodiments, the water reservoir has a size range of about 1 ml to about 100 ml. In some embodiments, the water reservoir includes a mechanical or electrical volume indicator so that the enclosed volume of liquid in the water reservoir is visible to a user. In some embodiments, the water reservoir has a transparent wall. In some embodiments, the water reservoir is sized to be in the range of about 5 mm³ to about 8 cm³. In some embodiments, the water reservoir includes an outlet. In some embodiments, the outlet is electrically controlled by a valve or mechanically controlled by a switch.

In some embodiments, the water reservoir includes a sensor for sensing one or more of saliva residuals, tooth paste residuals, proteins, bacteria, molecules, and microorganisms. In some embodiments, if the sensor valve does not meet a pre-determined threshold, a rinsing function is automatically activated until the sensor valve meets the pre-determined threshold. In some embodiments, if the sensor valve does not meet a pre-determined threshold, a saliva collection function is automatically disabled. In some embodiments, the water reservoir is replaceable.

### Buffer Reservoirs

In some embodiments, the systems, devices, and methods disclosed herein include a buffer reservoir or the like. In some embodiments, the buffer reservoir is enclosed in the handheld handle of the device or the base accommodating the device. In some embodiments, the buffer reservoir is directly in fluid communication with a detection unit and/or a valve. In some embodiments, the water reservoir is in direct or indirect fluid communication with a valve, a reversible fluidic connection, a suction tip, a saliva reservoir, a switch, a detection unit, a cartridge, an inlet, a tube fitting, and an outlet. In some embodiments, the water reservoir includes a pumping element so that liquid therewithin is pumped in or out of the water reservoir. In some embodiments, the buffer reservoir is made of material that is chemically inert to a buffer solution, water, saliva, and saliva rinsing solution. In some embodiments, the buffer reservoir is connected to a bi-directional pump.

In some embodiments, the buffer reservoir is electrically or mechanically controlled by at least one of a switch, a valve, and a reversible connection. In further embodiments, the buffer reservoir is individually controlled by the switch, the valve or the reversible connection to be in an open or a closed position.

In some embodiments, the buffer reservoir has a size range of about 0.1 ml to about 100 ml. In some embodiments, the buffer reservoir includes a mechanical or electrical volume indicator so that the enclosed volume of liquid in the reservoir is visible to a user. In some embodiments, the reservoir has a transparent wall. In some embodiments, the reservoir is sized to be in the range of about 5 mm³ to about 8 cm³. In some embodiments, the r reservoir includes an outlet. In some embodiments, the outlet is electrically controlled by a valve or mechanically controlled by a switch.

In some embodiments, the reservoir includes a sensor for sensing one or more of saliva residuals, tooth paste residuals, a buffer solution proteins, bacteria, molecules, and microorganisms.

### Reversible Connections

In some embodiments, the systems, devices, and methods disclosed herein include a reversible connection, connector, or the like. In some embodiments, the reversible connection is configurable at a connected position or a disconnection position. In some embodiments, the reversible connection is mechanically controlled to switch from one position to the other position. In some embodiments, the reversible connection is electrically controlled to switch from one position to the other position. In further embodiments, the reversible connection is controlled by a user selection at a user interface. In some embodiments, the reversible connection utilizes at least two magnets. The at least two magnets are located on the opposite sides to be connected by the reversible connection. In some cases, at least one magnet is located on the handheld device, and at least one magnet is located on the base accommodating the handheld device. the In certain embodiments, the reversible connection is used to fluidly connect any two selected from: the inlet, the first valve, the second valve, an internal tubing, a suction tip, filter, a saliva reservoir, a reversible fluidic connection, a tooth brush head, bristles, a base accommodating the hand-held device, a syringe, a piston, and a pump.

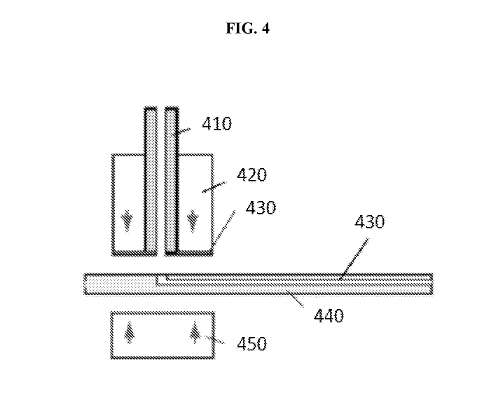

Referring to FIG. 4, in a particular embodiment, a reversible fluidic connection includes a first magnet 420 surround a tube or a needle 410 optionally at the bottom of a handheld portable device for saliva collection. In the same embodiment, the reversible fluidic connection includes a second magnet 450 optionally at a base accommodating the handheld portable device. In the same embodiments, the reversible fluidic connection includes a fluidic channel 460 in between the first magnet 420 and the second magnet 450. Optionally, the first magnet and second magnet attract each other and attaches to the opposite side of the fluid channel 460, thus, the fluid channel is connected to the tube or needle 410 fluidly. Optionally, a gasket 430 is between the mating surfaces, to prevent leakage. In the same embodiments, optionally, a chip 440 is placed in between the two magnets for actuation of magnetic attraction or repulsion.

### Handheld Handles

In some embodiments, the devices, systems, and methods disclosed herein include a handheld handle. In some embodiments, the handheld handle includes a saliva collector therewithin. In some embodiments, the handheld handle includes at least one connection interface configurable to connect to a suction tip. In some embodiments, the handheld handle includes an electrical control unit, a digital processing unit, a programmed microchip, a microprocessor, or use of the same. In some embodiments, the electrical control unit receives user input at the user interface. In some embodiments, the handheld handle includes a digital display. In some embodiments, the electrical control unit controls at least one element selected from: a bi-directional pump, a digital display, a user interface, an indicator, a timer, a valve, a saliva reservoir, a water reservoir, a suction tip, a toothbrush head, a UV light source, a power source, a reversible fluidic connection, a switch, a syringe, a dispenser, a detection unit, a base, an inlet/outlet, a buffer reservoir, a cartridge, and a wireless system. In some embodiments, the control unit controls at least one function or step selected from: saliva collection, saliva rinsing, saliva testing, saliva transportation, tooth brushing, bristle retraction, bristle advancement, or bristle disinfection. In some embodiments, the control unit controls at least one predetermined sequence of functions selected from: 1) saliva rinsing, and then saliva collection; 2) saliva rinsing, then saliva collection, and then tooth brushing; 3) bristle disinfection, then tooth brushing, then saliva rinsing, and then saliva collection; 4) tooth brushing, then saliva rinsing, and then saliva collection; 5) saliva rinsing, then saliva collection, then tooth brushing; and then bristle retraction; 6) saliva rinsing, then saliva collection, and then saliva testing; 7) saliva rinsing, then saliva collection, then saliva testing, and then tooth brushing; 8) bristle disinfection, then tooth brushing, then saliva rinsing, then saliva collection, and then saliva testing; 9) tooth brushing, then saliva rinsing, then saliva collection, and then saliva testing; 10) saliva rinsing, then saliva collection, then saliva testing, then tooth brushing; and then bristle retraction; 11) saliva rinsing, then saliva collection, then tooth brushing, and then saliva testing. In some embodiments, the control unit controls fluidic communication or electrical communication between at least two selected from: a bi-directional pump, a digital display, a user interface, an indicator, a timer, a valve, a saliva reservoir, a water reservoir, a suction tip, a toothbrush head, a UV light source, a power source, a reversible fluidic connection, a switch, a syringe, a dispenser, a detection unit, a base, an inlet/outlet, a buffer reservoir, a cartridge, a wireless system, an external digital device. In some embodiments, an electrical control unit, a microprocessor, and a digital processing device are interchangeable in the systems, devices, and methods disclosed herein. In some embodiments, an electrical control unit, a microprocessor, and a digital processing device are equivalent elements in the systems, devices, and methods disclosed herein.

### Toothbrush Heads

In some embodiments, the systems, devices, and methods disclosed herein include a toothbrush head or the like. In some embodiments, the toothbrush head includes an electrical or mechanical control of bristles” retraction. In some embodiments, the bristles are retracted mechanically by sliding a side bar longitudinal along the toothbrush. In some embodiments, the bristles are retracted electrically by selection at a user interface. In some embodiments, the bristles are retracted automatically after a period of bristle movement has ended. In some embodiments, the bristles are retracted automatically after a tooth brushing step. In some embodiments, the bristles are retracted automatically after a predetermined period of time. In some embodiments, the retracted bristles are automatically disinfected. In some embodiments, disinfection of the retracted is initiated with a selection at a user interface. In some embodiments, the retracted bristles are disinfected with an ultraviolet (UV) light source. In some embodiments, the retracted bristles are disinfected with at least one UV light-emitting diode (LED). In some embodiments, the UV light has a wavelength of around 260 nm, 254 nm, or 360 nm. In some embodiments, the UV light has a wavelength of from around 200 nm to 310 nm. In some embodiments, the retracted bristles are disinfected with a low-pressure mercury-vapor lamp. In some embodiments, the retracted bristles are disinfected with pulsed UV light. In some embodiments, the dose of the UV light is in the range of around 2 mJ/cm² to around 80 mJ/cm². In some embodiments, the bristles are retracted into a cavity within the surface of the toothbrush head. In some embodiments, a UV light source is located on the inner surface of the toothbrush head.

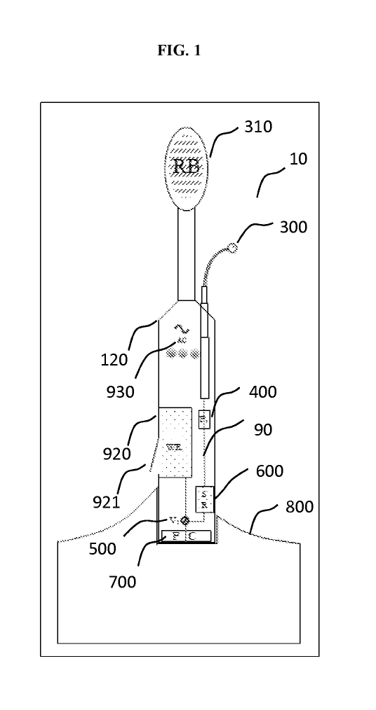

Referring to FIG. 1, in a particular embodiment, the handheld portable device 10 for saliva collection and tooth brushing includes a handheld handle 120 and a base 800 accommodating the handheld device for fluidic, electrical, and mechanical connections. In this embodiment, the device 10 includes a handheld handle 120 which is configurable to removably connect to a toothbrush head 310. In the same embodiment, the handle 120 is fluidly connectable to a suction tip 300 in a removable manner via at least one connection interface. In some embodiments, the connection interface to the toothbrush head and the suction tip is the same. In other embodiments, the connection interface to the toothbrush head and the suction tip is different and independent. In this embodiment, the suction tip 300 is fluidly connected directly to a bi-directional pump 400. In some embodiments, the pump 500 is a peristaltic pump. In the same embodiment, the pump 400 is directly connected to a saliva reservoir 600. In this embodiment, a valve 500 is fluidly connected to the saliva reservoir 600, a reversible fluidic connection 700, and a water reservoir 920. In some embodiments, the water reservoir 920 is located higher than the saliva reservoir 600 to facilitate liquid flow into the saliva reservoir 600 for rinsing purposes. In this particular embodiment, the valve 500 is electrically controlled to allow fluid communication between any two or all three elements that are fluidly connected to the valve. In some embodiments, these three elements are the saliva reservoir 600, the reversible fluidic connection 700, and the water reservoir 920. In this embodiment, the reversible fluidic connection 700 directly connects to the base 800. The water reservoir has an external filling lid 921, said lid is located higher than the contacting areas of the handle 120 to the base 800 so that water filling is not affected by the insertion of the handle 120 to the base 800. In the same embodiment, the handheld handle 120 includes a rechargeable power source 930. The fluidic connections between any two above-mentioned elements are via internal tubing 900.

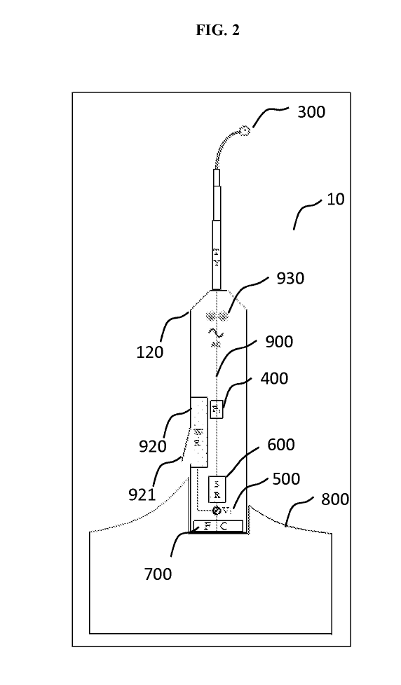

Referring to FIG. 2, in a particular embodiment, the handheld portable device 10 for saliva collection includes a handheld handle 120 and a base 800 accommodating the handheld device for fluidic, electrical, and mechanical connections. In the same embodiment, the handle 120 is fluidly connectable to a suction tip 300 in a removable manner via at least one connection interface. In some embodiments, the connection interface to an optional toothbrush head and the suction tip is the same. In other embodiments, the connection interface to the optional toothbrush head and the suction tip is different and independent. In this embodiment, the suction tip 300 is fluidly connected directly to a bi-directional pump 400. In some embodiments, the pump 500 is a peristaltic pump. In the same embodiment, the pump 400 is directly connected to a saliva reservoir 600. In this embodiment, a valve 500 is fluidly connected to the saliva reservoir 600, a reversible fluidic connection 700, and a water reservoir 920. In some embodiments, the water reservoir 920 is located higher than the saliva reservoir 600 to facilitate liquid flow into the saliva reservoir 600 for rinsing purposes. In this particular embodiment, the valve 500 is electrically controlled to allow fluid communication between any two or all three elements that are fluidly connected to the valve. In some embodiments, these three elements are the saliva reservoir 600, the reversible fluidic connection 700, and the water reservoir 920. In this embodiment, the reversible fluidic connection 700 directly connects to the base 800. The water reservoir has an external filling lid 921; said lid is located higher than the contacting areas of the handle 120 to the base 800 so that water filling is not affected by the insertion of the handle 120 to the base 800. In the same embodiment, the handheld handle 120 includes a rechargeable power source 930. The fluidic connections between any two above-mentioned elements are via internal tubing 900.

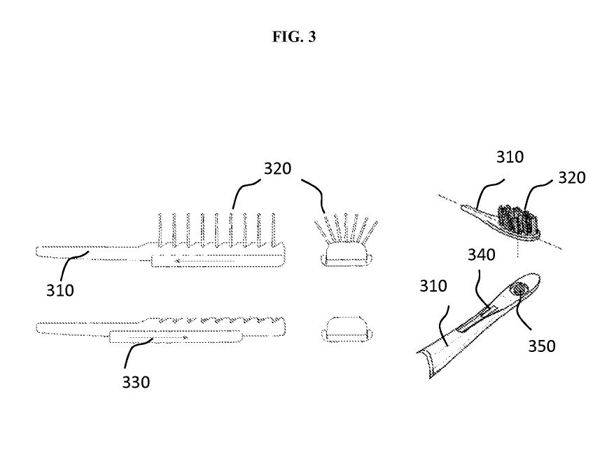

Referring to FIG. 3, in a particular embodiment, the bristles 320 of the toothbrush head 310 is retracted mechanically by sliding a side bar 330 longitudinally away from the tip of the tooth brush. In the same embodiment, the bristles are advanced out by sliding the slide bar 330 longitudinally in an opposite direction toward the tip of the toothbrush. In some embodiments, the bristles 310 are retracted using a slidable button 340 on the top surface of the tooth brush head 310. In this embodiment, a UV light LED 350 is located within the toothbrush head 310 and it is configurable to disinfect the bristles 320 when the bristles are retracted into the cavity of the toothbrush head.

### Diseases and Biomarkers

In certain embodiments, the systems, devices, and methods disclosed herein are used for testing at least one salivary biomarker. In some embodiments, a salivary biomarker include at least one selected from: glucose, fatty acid, nitric oxide, cortisol, c-reactive protein (CRP), secretory immunoglobulin A (IgA), solute carrier family 6 (SLC6A4), 5-hydroxytryptamine-2C (5HT2C), gated calcium channel, ankyrin G, a1-anti-trypsin, brain-derived neurotrophic factor (BDNF), calcium binding protein B (S100B), myelo-peroxidase, soluble tumor necrosis factor receptor type II (s-TNFRII), epidermal growth factor (EGF), prolactin, resistin, senescence-associated secretory phenotype (SASP), spermidine/spermine N1-acetyltransferase (1SAT1), phosphatase and tensin homolog (PTEN), myristoylated alanine-rich C-kinase substrate (MARCKS), mitogen-activated protein kinase-kinase-kinase-3 (MAP3K3), signal transducer cluster of differentiation 24/heat stable antigen (CD24/HSA), forkhead box protein N3 (FOXN3), phosphoinositide-3-kinase, regulatory subunit 5 (PIK3R5), guanine nucleotide-binding protein 1 (GBP1), calcitonin gene related peptide (CGRP), estrogen, vasoactive intestinal peptide VIP, alpha-amylase, prostaglandin E2 (PGE2), prostacyclin (PGI2), β-endorphin, corticotropin-releasing hormone (CRH), dehydroepiandrosterone (DHEA), dehydroepiandrosterone-sulfate (DHEA-S), estradiol, interleukin-lbeta (IL-1β), interleukin 2 (IL-2), interleukin 4 (IL-4), interleukin 5 (IL-5), interleukin 6 (IL-6), interleukin 9 (IL-9), interleukin 17 (IL-17), progesterone, tumor necrosis factor alpha (TNF-α); cardiac troponin I (cTnI) cardiac troponin T (cTnT), creatine phosphokinase-MB (CK-MB), brain natriuretic peptide (BNP), N-terminal pro-brain natriuretic peptide (NT-proBNP); myoglobin, matrix metallopeptidase 9 (MMP-9), TNF receptor superfamily member 5 (CD40), soluble intercellular adhesion molecule-1 (sICAM-1), breast cancer 1 and 2 (BRCA1&2), receptor tyrosine-protein kinase erbB-2/human epidermal growth factor receptor2 (C-erbB-2/HER2/neu), cancer antigen 15-3 (CA15-3), mucin 1, cathepsin D, p53, homeodomain-containing transcription factor NKX3.1, zinc-alpha-2-glycoprotein, calprotectin (S100A8, S100A9), anaplastic lymphoma kinase (ALK), epidermal growth factor receptor (EGFR), V-Ki-ras2 Kirsten rat sarcoma viral oncogene homolog (KRAS), v-Raf murine sarcoma viral oncogene homolog B (braf V600E), carcinoembryonic antigen (CEA), uridine diphosphate glucuronyltransferase 1-1 (UGT1-A1), 3-methylhistidine, glucose-1-phosphate, glucose-6-phosphate, taurine, chromogranin A, melatonin, testosterone, 1,5-anhydroglucitol, chromogranin A, oxido-reductase; glycated hemoglobin (HbAlc), uric acid, transaldolase, phosphoglycerate mutase I, luteinizing hormone (LH), estrone-3-glucuronide (E3G), follicle stimulating hormone (FSH), follicle stimulating hormone (FSH), Androstenedione, Estradiol (E2), Estriol (E3), 17α-hydroxyprogesterone, androstenedione, estrogen receptors, estrone glucuronide (E1G) pregnanediol glucuronide (PdG), androstenedione, insulin-like growth factor 1 (IGF-1), tau, amyloid beta, alpha-synuclein, parkinson protein 7 (PARK7), human leucocyte antigens (HLAs) type II, fractalkine.

In certain embodiments, the systems, devices, and methods disclosed herein are used for routine tracking of physiological conditions, healthy as well as disease conditions, based on monitoring of known salivary biomarkers of said physiological or pathological conditions. In some embodiments, a physiological or pathological condition include at least one selected from: sleep quality, physical, fertility-related conditions, oral-related diseases, bone loss, nutrition, autoimmune diseases, sexually transmitted diseases, cardiovascular health, systemic diseases, psychological health, and common cancer types. In some embodiments, t a physiological or pathological condition include at least one selected from: sleep apnea, sleep deprivation, jet lag recovery, fatigue, overtraining syndrome, ovulation, pregnancy, menopause, periodontal diseases, gingivitis, oral carcinoma, squamous cell carcinoma, oral squamous cell carcinoma (OSCC), osteoporosis and alveolar remodeling, glucose levels, metabolic/anabolic status, eating disorders, rheumatoid arthritis, Sjogren's syndrome and benign lymphoepithelial lesions, acute myocardial infarction, diabetes, fibromyalgia, gout, mood disorder, depression, suicidality, anxiety, stress, breast cancer, prostate cancer, pancreatic cancer, melanoma, lymphoma, brain cancer, bone marrow cancer, human immunodeficiency virus (HIV), human papilloma virus (HPV), cervical cancer, ovary cancer, pancreatic cancer, esophagus cancer, stomach cancer, lung-and-bronchus cancer, and colorectal cancers, oral cancer, hepatitis, parasitic infection, helicobacter pylori infection, periodontitis, and allergy.

### Bases

In some embodiments, the devices, systems, and methods disclosed herein include a base to accommodate the toothbrush. In some embodiments, the base is fluidly connected to the portable handheld device. In some embodiments, the base includes a cavity for accommodating at least one disposable cartridge. In some embodiments, the base includes a signal detection cell configurable to capture or detect different salivary biomarkers. In some embodiments, the base includes at least one data transmitting element from the detection cell to a microprocessor. In some embodiments, the base has a microfluidics system comprising at least one biosensor. In some embodiments, the microfluidics system handles saliva or liquid delivery from the handheld device to the detection cell, where the salivary biomarkers are captured and analyzed. In some embodiments, the biosensor is an optical sensor, a motion sensor, a temperature sensor, a Micro-electromechanical system (MEMS) sensor, an electrical sensor, a chemical sensor, a PH sensor, a colorimetric sensor, a sound sensor, an electrochemical sensor, or a pressure sensor. In some embodiment, the base includes a microprocessor for quantitative or qualitative data analysis based on the data generated form the detection cell. In some embodiments, the base has a wireless connection for transmitting data to or receiving data from a remote location. In some embodiments, the base is connected to the portable handheld device via a reversible fluidic connection or a valve. In some embodiments, the base includes a fluid inlet/outlet. In some embodiments, the base includes a fluid dispenser. In some embodiments, the bases include a saliva reservoir and/or a buffer reservoir. In some embodiments, the base includes a pump. In some embodiments, the base includes a valve. In some embodiments, the base has an electrical charging component in order to charge the rechargeable power source of the handheld device. In some embodiments, the base includes internal tubing fluidly connecting one or more selected from a detection cell, an inlet/outlet, a pump, a reservoir, a valve, a sensor, a cartridge, and a buffer reservoir.

In some embodiments, the base includes a reversible fluidic connection. In some embodiments, fluidic communication between the handheld handle and the base is established when the reversible fluidic connection of the base and the reversible fluidic connection of the handheld device are both in the connected position. In some embodiments, fluidic communication between the handheld handle and the base is established when the handheld handle is properly placed back in the base. In some embodiments, the reversible fluidic connection of the base and the reversible fluidic connection of the handheld device are automatically configured to the connected position when the handheld handle is properly placed back in the base. In some embodiments, the reversible fluidic connection of the base and the reversible fluidic connection of the handheld device are configured to the connected position via at least one electrical control unit. In some embodiments, fluidic communication between the handheld handle and the base is established when the reversible fluidic connection of the base and the reversible fluidic connection of the handheld device are both in the connected position and when the handheld handle is properly placed back in the base. In some embodiments, the base includes a pump for pumping at least one of a buffer, a reagent, an untested saliva sample, and a mixture of liquid in the detection cell. In some embodiments, the pumping force of the pump at the base is used to cleanse at least one of an internal tube, a detection unit, an outlet, and a dispenser located therewithin. In some embodiments, the cartridge is configurable to dispose fluid from the detection unit. In some embodiments, the cartridge is configured to dispose fluid from at least one internal tube in the base.

### Detection Units

In certain embodiments, the systems, devices, and methods disclosed herein include a detection unit, a detection cell, or use of the like. In some embodiments, for saliva testing, at least a saliva sample, a buffer solution, and a reagent is mixed in the detection cell for detection of at least one salivary biomarker. In some embodiments, a detection cell is rinsed by water or a rinsing solution after each independent testing session. In some embodiments, a detection cell includes at least a biosensor for sensing signal of a salivary biomarker. In some embodiments, the sensing signal is transmitted to a processor enclosed within the base for signal analysis and data quantification. In some embodiments, the detection cell is electrically controlled. In some embodiments, the detection cell is controlled by a saliva testing function/step of the device. In some embodiments, the detection unit is fluidly connected to an outlet or a waste reservoir for disposal of tested saliva samples or rinsing solutions.

### Electronics

In certain embodiments, the systems, devices, and methods disclosed herein include electronics, a circuit board, or the like. In some embodiments, the device includes one or more selected from: an electrical motor, an electrical control unit, a user interface, an integrated circuit, a circuit chip, a circuit board, an electrical timer, a rechargeable battery, a piezoelectric transducer, an electrical switch, an induction coil, a resistor, and LED, a diode, a semiconductor, an electrical display, a gear, programmable MEMS chips, a programmable logic chip, an electrode, a sensor, a resistor, a coil, a conductor, an inductor, a voltage generator, a current generator, a capacitor, an antenna, and a cam.

### User Interfaces

In certain embodiments, the systems, devices, and methods disclosed herein include a user interface or the like. In some embodiments, the user interface allows user selection of one or more functions selected from: a user interface configured to allow user selection of at least one function selected from: saliva collection, saliva ejection, saliva rinsing, tooth brushing, bristle retraction, bristle advancement, or bristle disinfection. In some embodiments, the user interface allows user selection of one preprogrammed sequence of functions selected from: 1) saliva rinsing, and then saliva collection; 2) saliva rinsing, then saliva collection, and then tooth brushing; 3) bristle disinfection, then tooth brushing, then saliva rinsing, and then saliva collection; 4) tooth brushing, then saliva rinsing, and then saliva collection; 5) saliva rinsing, then saliva collection, then tooth brushing; and then bristle retraction; 6) saliva rinsing, then saliva collection, and then saliva testing; 7) saliva rinsing, then saliva collection, then saliva testing, and then tooth brushing; 8) bristle disinfection, then tooth brushing, then saliva rinsing, then saliva collection, and then saliva testing; 9) tooth brushing, then saliva rinsing, then saliva collection, and then saliva testing; 10) saliva rinsing, then saliva collection, then saliva testing, then tooth brushing; and then bristle retraction; 11) saliva rinsing, then saliva collection, then tooth brushing, and then saliva testing. In some embodiments, the user interface includes an on/off switch. In some embodiments, the user interface includes a function selection button and/or a button for selecting a sequence of functions. In some embodiments, the user interface includes a button for sensing contamination level within the handheld device. In some embodiments, the user interface includes a button for setting a timer. In some embodiments, the user interface includes an indicator for one or more selected from: changing a filter, replacing the toothbrush head, replacing the saliva reservoir, changing the suction tip, cleaning the reservoir, cleaning the inlet, cleaning the suction tip, cleaning the internal tubing, cleaning the reversible fluidic connection, cleaning the base, cleaning the detection cell, and cleaning the outlet. In some embodiments, the user interface includes an indicator of an error. In further embodiments, the indicator provides information regarding the function, the elements, or the cause of the error. In some embodiments, the user interface includes a digital display for presenting system messages, testing results, user selections, timer, and/or other related information to a user.

### Inlets/Outlets

In some embodiments, the systems, devices, and methods disclosed herein include an inlet/outlet for liquids passage. In some embodiments, the inlet is electrically controlled by a valve or mechanically controlled by a switch. In some embodiments, the inlet/outlet is reversibly connected to the rest of the device via a connection interface. In some embodiments, the inlets and outlets are replaceable.

### Digital Processing Devices

In some embodiments, the platforms, media, methods and applications described herein include a digital processing device, a processor, a microprocessor, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, media, methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, media, methods and applications described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™ and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of barcode, route, parcel, user, or network information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### Web Browser Plug-in

In some embodiments, the computer program includes a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

1-39. (canceled)

40. A portable handheld device for tooth brushing and saliva collection for daily monitoring of salivary biomarkers of physiological conditions of a subject, the device comprising:
a) a handheld handle comprising a saliva collector, the saliva collector comprising:
i) a suction tip;
ii) a bi-directional pump electrically configurable for pumping towards two opposite directions;
iii) a saliva reservoir;
iv) a valve;
v) a water reservoir, the water reservoir comprising an external filling lid;
vi) a reversible fluidic connection configurable in a connected position and a disconnected position; and
b) a base accommodating the handheld handle, the base configurable to fluidly communicate with the handheld handle through at least the reversible fluidic connection, wherein the valve is electrically configured to allow fluid communication directly between at least two elements selected from: the saliva reservoir, the water reservoir, and the reversible fluidic connection, and wherein the bi-directional pump is electrically configurable to allow fluid communication directly between the suction tip and the saliva reservoir.

41. The device of claim 40 comprises a toothbrush comprising:
a) a toothbrush head comprising a plurality of bristles, wherein the toothbrush head is configured to retract the plurality of bristles; and
b) a UV light source configured to disinfect the retracted plurality of bristles.

42-47. (canceled)

48. The device of claim 40, wherein the direct fluidic communication is through at least one internal tube.

49. The device of claim 40, wherein the direct fluidic communication is bi-directional.

50-63. (canceled)

64. The device of claim 40 comprises a user interface configured to allow user selection of at least one function selected from: saliva collection, saliva rinsing, saliva transportation, saliva testing, tooth brushing, bristle retraction, bristle advancement, or bristle disinfection.

65-66. (canceled)

67. The device of claim 40, wherein the handheld handle comprises at least one connection interface configurable to connect to the toothbrush head.

68. The device of claim 40, wherein the base is fluidly connected to the handheld handle automatically via the reversible fluidic connection when the handheld handle is properly placed back in the base and a predetermined condition is met.

69. The device of claim 40, wherein the base is electrically connected to the device automatically when the handheld handle is properly placed back in the base.

70. (canceled)

71. The device of claim 40, wherein the base comprises:
h) a second reversible fluidic connection configurable in a connected position and a disconnected position;
i) a detection unit configurable to fluidly communicate with the second reversible fluidic connection;
j) a buffer reservoir configurable to fluidly communicate with the detection unit;
k) a second electrical pump;
l) a port configurable to hold a removable cartridge and allow fluidic connection of the removable cartridge to the detection unit;
m) a microprocessor; and
n) a wireless system,
wherein the second reversible fluidic connection is configured to allow fluidic connection to the handheld handle.

72. The device of claim 71, wherein the base is fluidly connected to the handheld handle when the second reversible fluidic connection and the reversible fluidic connection of the handheld handle are in the connected position.

73. The device of claim 71, wherein the removable cartridge is configurable to hold at least one reagent therewithin.

74. The device of claim 71, wherein the wireless system is configurable to transmit data to or receive data from at least one external source.

75. (canceled)

76. The device of claim 40, wherein the device is configurable to monitor at least one salivary biomarker for at least one physiological condition in at least one category selected from: sleep quality, physical performance, fertility-related conditions, oral-related diseases, bone, nutrition, autoimmune diseases, cardiovascular health systemic diseases, psychological health, and common cancer types.

77. The device of claim 40, wherein the device is configurable to monitor at least one salivary biomarker for at least one physiological condition selected from: sleep apnea, sleep deprivation, jet lag recovery, fatigue, overtraining syndrome, ovulation, pregnancy, menopause, gingivitis, oral carcinoma, squamous cell carcinoma, OSCC, osteoporosis, alveolar remodeling, glucose abnormalities, metabolic/anabolic status, eating disorders, rheumatoid arthritis, Sjogren's syndrome, lymphoepithelial lesion, acute myocardial infarction, diabetes, fibromyalgia, gout, mood disorder, depression, suicidality, anxiety, stress, breast cancer, prostate cancer, pancreatic cancer, lung- and bronchus cancer, and colorectal cancer.

78. The device of claim 40, wherein the device is configurable to monitor at least one salivary biomarker selected from: glucose, fatty acid, nitric oxide, Cortisol, c-reactive protein (CRP), secretory immunoglobulin A (IgA), solute carrier family 6 (SLC6A4), 5-hydroxytryptamine-2C (5HT2C), gated calcium channel, ankyrin G, a1-anti-trypsin, brain-derived neurotrophic factor (BDNF), calcium binding protein B (S100B), myelo-peroxidase, soluble tumor necrosis factor receptor type II (s-TNFRII), epidermal growth factor (EGF), prolactin, resistin, senescence associated secretory phenotype (SASP), spermidine/spermine N1-acetyltransferase (1SAT1), phosphatase and tensin homolog (PTEN), myristoylated alanine-rich C-kinase substrate (MARCKS), mitogen-activated protein kinase-kinase-kinase-3 (MAP3K3), signal transducer cluster of differentiation 24/heat stable antigen (CD24/HSA), forkhead box protein N3 FOXN3), phosphoinositide-3-kinase, regulatory subunit 5 (PIK3R5), guanine nucleotide-binding protein 1 (GBP1), calcitonin gene related peptide (CGRP), estrogen, vasoactive intestinal peptide YIP, alphaamylase, prostaglandin E2 (PGE2), prostacyclin (PGI2), P-endorphin, corticotropin-releasing hormone (CRH), dehydroepiandrosterone (DHEA), dehydroepiandrosterone-sulfate (DHEA-S), estradiol, interleukin-1 beta (IL-ip), interleukin 2 (TL-2), interleukin 4 (IL-4), interleukin 5 (IL-5), interleukin 6 (IL-6), interleukin 9 (IL-9), interleukin 17 (IL-17), progesterone, tumor necrosis factor alpha (TNF-α); cardiac troponin 1 (cTn1) cardiac troponin T (cTnT), creatine phosphokinase-MB (CK-MB), brain natriuretic peptide (BNP), N-terminal pro-brain natriuretic peptide (NT-proBNP); myoglobin, matrix metallopeptidase 9 (MMP-9), TNF receptor superfamily member 5 (CD40), soluble intercellular adhesion molecule-1 (sICAM-1), breast cancer 1 and 2 (BRCA1&2), receptor tyrosine-protein kinase erbB-2/human epidermal growth factor receptor2 (C-erbB-2/HER2/neu), cancer antigen 15-3 (CA15-3), mucin 1, cathepsin D, p53, homeodomain-containing transcription factor NKX3.1, zinc-alpha-2-glycoprotein, calprotectin (S100A8, S100A9), anaplastic lymphoma kinase (ALK), epidermal growth factor receptor (EGFR), V-Ki-ras2 Kirsten rat sarcoma viral oncogene homolog (KRAS), v-Raf murine sarcoma viral oncogene homolog B (braf Y600E), carcinoembryonic antigen (CEA), uridine diphosphate glucuronyltransferase 1-1 (UGT1-A1), 3-methylhistidine, glucose-1-phosphate, glucose-6-phosphate, taurine, chromogranin A, melatonin, testosterone, 1,5-anhydroglucitol, chromogranin A, oxido-reductase; glycated hemoglobin (HbAlc), uric acid, transaldolase, phosphoglycerate mutase I, luteinizing hormone (LH), estrone-3-glucuronide (E3G), follicle stimulating hormone (FSH), androstenedione, estradiol (E2), estriol (E3), 17α-hydroxyprogesterone, androstenedione, estrogen receptors, estrone glucuronide (E1G) pregnanediol glucuronide (PdG), androstenedione, insulin-like growth factor 1 (IGF-1), tau, amyloid beta, alpha-synuclein, parkinson protein 7 (PARK7), human leucocyte antigens (HLAs) type II, fractalkine.

79. A method of saliva collection using the device of claim 40 for daily monitoring of at salivary biomarkers of physiological conditions of a subject, the method comprising:
a) rinsing a handheld handle of the device using a bi-directional pump using water or a rinsing solution from a water reservoir, the handheld handle of the device being properly detached from a base accommodating the device, wherein a valve is in an open position and a reversible fluidic connection is in a connected position;
b) collecting a saliva sample from a mouth of the subject through a suction tip to a saliva reservoir; and
c) transporting the saliva sample from the saliva reservoir to the base, the handheld handle being properly placed back in the base and the fluidic connection between the handheld handle and the base being electrically established, wherein the valve is in the open position and the reversible fluidic connection is in the connected position;
wherein the valve is electrically configured to allow fluid communication directly between at least two selected from: the saliva reservoir, the water reservoir, and the reversible fluidic connection, and
wherein the bi-directional pump is electrically configurable to allow fluid communication directly between the suction tip and the saliva reservoir, and wherein the water reservoir comprises an external filling lid.

80-109. (canceled)
